(19) United States
(12) Patent Application Publication
(10) Pub. No.: US 2009/0165167 A1
(43) Pub. Date: Jun. 25, 2009

(54) Means and Methods to Enhance the Production of Vinblastine and Vincristine in Catharanthus Roseus

(76) Inventors: Alain Goossens, Lokeren (BE); Heiko Rischer, Espoo (FI); Dirk G. Inze, Moorsel-Aalst (BE); Matej Oresic, Espoo (FI); Kirsi-Marja Oksman-Caldentey, Helsinki (FI)

Correspondence Address: TRASKBRITT, P.C., P.O. BOX 2550, SALT LAKE CITY, UT, 84110, US

(21) Appl. No.: 11/992,030

(22) PCT Filed: Sep. 14, 2006
(86) PCT No.: PCT/EP2006/066366

§ 371 Date: Jun. 26, 2008

(30) Foreign Application Priority Data
Sep. 14, 2005 (EP) 05108437.4
Feb. 16, 2006 (EP) 06110006.1

(51) Int. Cl. A01H 1/00; C07H 21/00; C12N 15/74; C12N 5/10

(52) U.S. Cl. 800/278; 536/23.1; 435/320.1; 435/410

(57) ABSTRACT

The present invention relates to the field of plant secondary metabolites, particularly terpenoid indole alkaloids. More particularly the present invention relates to the production of the anti-cancer metabolites vincristine and vinblastine. The invention provides novel polynucleotide sequences derived from Catharanthus roseus and the use of said polynucleotide sequences to stimulate the production of vinblastine and/or vincristine in plants of Catharanthus roseus and plant cell lines derived thereof. The invention further relates to recombinant plants, plant cells, tissue and organ cultures obtainable by the process of the invention.

### FIELD OF THE INVENTION

The present invention relates to the field of plant secondary metabolites, particularly terpenoid indole alkaloids. More particularly the present invention relates to the production of the anti-cancer metabolites vincristine and vinblastine. The invention provides novel polynucleotide sequences derived from Catharanthus roseus and the use of said polynucleotide sequences to stimulate the production of vinblastine and/or vincristine in plants of Catharanthus roseus and plant cell lines derived thereof. The invention further relates to recombinant plants, plant cells, tissue and organ cultures obtainable by the process of the invention.

### BACKGROUND OF THE INVENTION

The medicinal plant Catharanthus roseus L. G. Don has been of enormous interest to pharmaceutical science because it contains more than 120 terpenoid indole alkaloids (TIAs) some of which exhibit potent pharmacological activities (van der Heijden et al., 2004). The antihypertensive alkaloid ajmalicine and the antineoplastic bisindole alkaloids vinblastine and vincristine are in clinical use. The latter two anticancer compounds are produced only in very low amounts in the plants (Noble, 1990) and their total synthesis is not economically feasible. These compounds are consequently still directly isolated from field-grown plants. Alternatively, derivatives such as vinorelbine are synthesized from plant-derived building blocks (van der Heijden et al., 2004). As a result of the costly extraction process and the influence of various environmental factors on harvest yields, the market prices for vinblastine and vincristine are exorbitantly high (Moreno et al., 1995).

Despite significant efforts, cell cultures have not offered a valid production alternative so far. Engineering plant metabolic pathways in order to increase the productivity e.g. in cultivated plant cells is generally a difficult task because our fundamental knowledge of the biosyntheses is far from complete even in the well studied example of the TIA pathway. General problems encountered when characterizing plant metabolomes are the enormous chemical diversity of the compounds (primary and secondary) and their highly complex nature (Hirai et al., 2004; Oksman-Caldentey and Inzé, 2004). Undifferentiated Catharanthus cells can produce fairly high levels of alkaloids such as ajmalicine, tabersonine and catharanthine but not of vindoline, the compound that together with catharanthine constitutes the building blocks for the in vivo formation of bisindole alkaloids (FIG. 1). All TIAs in C. roseus are derived from the central precursor strictosidine, which is a fusion product of the shikimate pathway-derived tryptamine moiety and of the plastidic non-mevalonate (MEP) pathway-derived secologanin moiety (FIG. 1). Starting from the amino acid tryptophan and the monoterpenoid geraniol, the biosynthesis of C. roseus bisindole alkaloids involves at least 35 intermediates and 30 enzymes or biosynthetic genes (St-Pierre et al., 1999; van der Heijden et al., 2004). The TIA biosynthetic pathway is under strict developmental and environmental control. For instance, expression of desacetoxyvindoline 4-hydroxylase (D4H) and vindoline accumulation is affected both by light and methyl jasmonate (MeJA) in C. roseus seedlings (Vazquez-Flota and De Luca, 1998), whereas TIA production in C. roseus cell cultures can be induced by various phytohormones, and biotic and abiotic elicitors (van der Heijden et al., 2004; Yahia et al., 1998 and references therein). Ultimately, transcription factors are responsible to coordinate the expression of biosynthetic genes in response to these external and internal signals. Members of the plant specific AP2/ERF transcription factor family have been identified in C. roseus, namely ORCA2 and 3 (Octadecanoid-Responsive Catharanthus AP2/ERF-domain transcription factor), the expression of which is induced by MeJA. ORCA proteins control the transcription of genes involved in the TIA biosynthesis such as strictosidine synthase (STR) by specific binding to a promoter element involved in jasmonate- and elicitor-responsive gene expression (JERE) (Menke et al., 1999; van der Fits and Memelink, 2001). Several other C. roseus transcriptional regulators have been identified, all displaying binding activity to the STR promoter (Chatel et al., 2003; Pauw et al., 2004; Siberil et al., 2001; van der Fits and Memelink, 2000), pointing to a considerable degree of complexity in the control of TIA biosynthesis. A deeper understanding of the regulatory system governing TIA metabolism is therefore of particular interest, in order to eventually allow successful metabolic engineering of alkaloid biosynthesis. Genome-wide cDNA-amplified fragment length polymorphism-based transcript profiling combined with metabolic profiling of elicited C. roseus cell cultures yielded an ample collection of known and novel C. roseus metabolites and transcript tags, centred on terpenoid indole alkaloids. Correlation network analysis between the expression profiles of the 417 gene tags and the accumulation profiles of 178 metabolite peaks allowed visualising novel gene-to-gene and gene-to-metabolite networks. This revealed the existence of divergent hormonal regulation, not only for the different branches of terpenoid indole alkaloid biosynthesis but also for various other periwinkle metabolic pathways. Simultaneously, the constructed correlation networks allowed distinguishing the genes and metabolites that are related to the biosynthesis of terpenoid indole alkaloids from those that do not. In the present invention a set of 66 genes were identified that can be used to enhance the production of tabersonine and/or catharanthine and/or vinblastine and/or vincristine.

### FIGURES

FIG. 1. Biosynthesis of C. roseus terpenoid indole alkaloids. (a) Formation of cathenamine. (b) TIA formation. Metabolites are written as full names in small-case and enzymes as abbreviations in capital letters. Full and dashed arrows indicate single and multiple conversion steps between intermediates, respectively. In the upper left corner transcription factors binding promoters of TIA biosynthetic genes are indicated. Additionally a graphical snapshot representing the relative transcript (boxes) and metabolite (circles) accumulation levels of samples harvested 12 h after elicitation is presented. The left and right boxes or circles reflect the influence of jasmonate and auxin, respectively. Red, induced expression compared to control without the phytohormone; green, repressed expression compared to control without the phytohormone; white, no effect of the phytohormone on expression; crossed, no transcript or metabolite accumulation detected. Enzymes and transcription factors listed: ORCA (octadecanoid-responsive Catharanthus AP2-domain), BPF (box P-binding factor), GBF (G-box binding factor), ZCT (zinc finger Catharanthus transcription factor), DXS (1-deoxy-D-xylulose-5-phosphate synthase), DXR (1-deoxy-D-xylulose-5-phosphate reductoisomerase), CMS (4-diphosphocytidyl-2C-methyl-D-erythrol 4-phosphate synthase), CMK (4-diphosphocytidyl-2C-methyl-D-erythrol kinase), MECS (2C-methyl-D-erythrol-2,4-cyclodiphosphate synthase), HDS (GCPE, 1-hydroxy-2-methyl-2-(E)-butenyl 4-diphosphate synthase), HDR (1-hydroxy-2-methyl-2-(E)-butenyl 4-diphosphate reductase), IPPI (Isopentenylpyrophosphate isomerase), G10H (geraniol 10-hydroxylase), CPR (cytochrome P450 reductase), 10HGO (10-hydroxygeraniol oxidoreductase), SLS (secologanin synthase), STR (strictosidine synthase), SGD (strictosidine β-D-glucosidase), AS (anthranilate synthase), TDC (tryptophan decarboxylase), T16H (tabersonine 16-hydroxylase), OMT (O-methyltransferase), NMT (N-methyltransferase), D4H (desacetoxyvindoline 4-hydroxylase), DAT (deacetylvindoline 4-O-acetyltransferase), MAT (acetyl-CoA:minovincine-O-acetyltransferase).

### AIMS AND DETAILED DESCRIPTION OF THE INVENTION

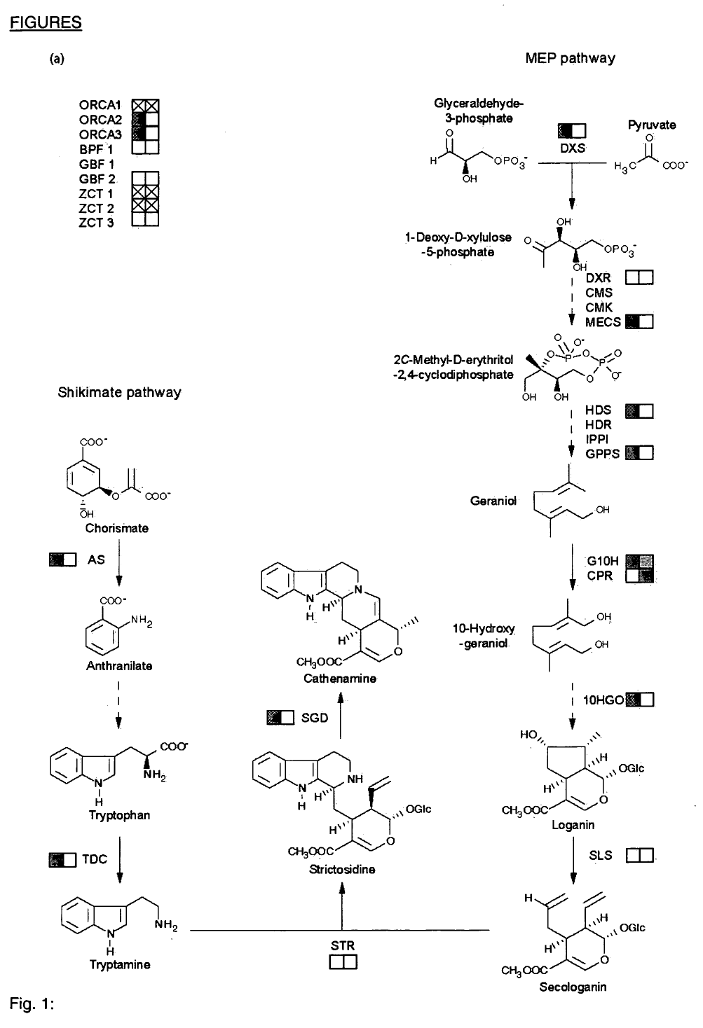

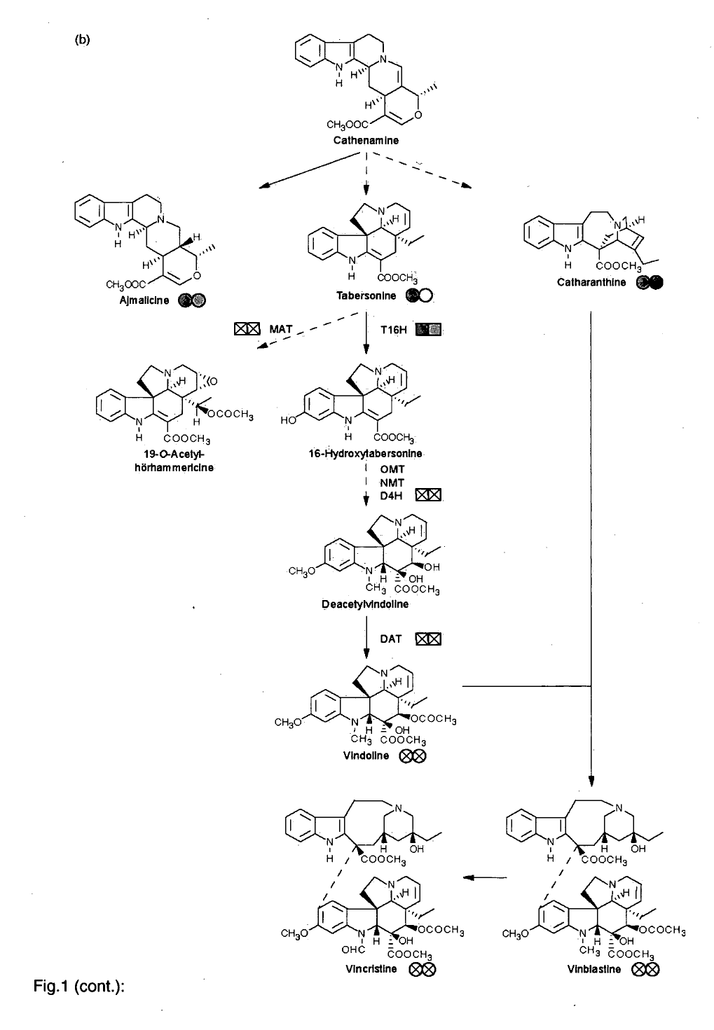

The regulatory and metabolic pathways determining the biosynthesis of biologically active plant compounds are generally poorly understood. This greatly impedes rational engineering of complicated metabolic networks involved in the production of an interesting compound. Whereas comprehensive genome-wide functional genomics approaches are successfully applied for holistic systems analysis of a select number of model plants like Arabidopsis, such an approach has not yet been reported for non-model plants representing most if not all medicinal plants. Large-scale gene discovery programs in medicinal plants such as Catharanthus roseus are enormously hampered by the fact that standard transcript profiling methods such as SAGE (Serial Analysis of Gene Expression) or micro-array analysis are not applicable because large sequence repertoires are not available for these non-model plant systems. However, the cDNA-AFLP technology offers the possibility to allow gene identification in such plants species and to acquire quantitative expression profiles at the same time (Goossens et al., 2003). In the present invention genome-wide cDNA-AFLP-based transcript profiling based on jasmonate-elicited C. roseus cell suspensions, allowed to build a substantial collection of known and novel genes of this medicinally important plant. Homology searches with the sequences from the C. roseus cDNA-AFLP tags revealed that 37% of these tags displayed no sequence similarity to any of the known plant genes. This finding was not unexpected considering the scarce availability of gene sequences from either C. roseus or other species belonging to the Apocynaceae family—that can be used for tag sequence extension in the BLAST searches—as compared to the abundant EST collections of tobacco, tomato and other Solanaceae species. For instance, when blasting the 417 nucleotide sequences of our cDNA-AFLP tags with the 236 prior to this study publicly available C. roseus EMBL entries, less than 10% gave a (near) perfect match. Thus, the vast majority of the tags identified here confer novel sequence information to C. roseus. When overlaying the TIA gene expression profiles with the TIA metabolite accumulation profiles on the pathway presented in FIG. 1, our findings clearly extend the earlier observations on the coordinate regulation of TDC and STR by MeJA in Catharanthus cells (St-Pierre et al., 1999) to the whole TIA pathway (up to 16-hydroxytabersonine).

Integration of genome-wide transcript and metabolite profiles allowed not only to visualise most of the known genes involved in TIA biosynthesis in a single experiment but also to draw novel gene-to-gene and gene-to-metabolite networks. As such, this invention provides 66 new genes which can be used to stimulate the production of tabersonine and/or catharanthine and/or vinblastine and/or vincristine in Catharanthus roseus plants and plant cell lines derived thereof.

Thus in a first embodiment the invention provides an isolated polynucleotide that enhances the production of tabersonine and/or catharanthine and/or vinblastine and/or vincristine in Catharanthus roseus, or plant cell lines derived thereof, that is selected from the group comprising SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66 or any variant or functional fragment thereof.

In yet another embodiment the invention provides a polynucleotide that enhances the production of tabersonine and/or catharanthine and/or vinblastine and/or vincristine in Catharanthus roseus, or plant cell lines derived thereof, wherein said polynucleotide shares at least 70% homology with a polynucleotide that is selected from the group comprising SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66 or any variant or functional fragment thereof.

In the present invention the sequences depicted in SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66 represent gene tags and full length sequences which are derived from the plant Catharanthus roseus. In particular SEQ ID NO: 49, 50, 53, 62, 63, 64 and 65 represent full length sequences. It is obvious for the person skilled in the art to isolate the complete gene by means of routine cloning methods based on the sequence information of the herein identified gene tags. One method which is non-limiting for isolating the complete gene or cDNA sequence is based on the use of said gene tags in DNA hybridization. In such a typical experiment a gene tag is labeled (e.g. fluorescent of radioactive) and used to fish out the corresponding nucleotide sequence from a library (e.g. an arrayed cDNA library). Another non-limiting method is the PCR RACE technology which is a popular method to get the sequences of the 5′ and 3′ ends of a mRNA via PCR amplification of the corresponding cDNA ends.

As used herein, the word “polynucleotide” may be interpreted to mean the DNA and cDNA sequence as detailed by Yoshikai et al. (1990) Gene 87:257, with or without a promoter DNA sequence as described by Salbaum et al. (1988) EMBO J. 7(9):2807.

As used herein, “fragment” refers to a polynucleotide of at least about 30 base pairs, typically 50 to 75, or more base pairs. If desired, the fragment may be fused at either terminus to additional base pairs, which may number from 1 to 20, typically 50 to 100, but up to 250 to 500 or more. A “functional fragment” means a polynucleotide fragment possessing the biological property able to enhance the production of tabersonine and/or catharanthine and/or vinblastine and/or vincristine in Catharanthus roseus or cell derived thereof. The term ‘production’ includes intracellular production and secretion into the medium. The term ‘enhances’ refers to an increase. With said enhancement or increase in the production of tabersonine and/or catharanthine and/or vinblastine and/or vincristine it is understood that said production may be enhanced by at least 20%, 30%, 40%, 50%, 60%, 70%, 80%, 90% or at least 100% relative to the untransformed plant or plant cell which was used to transform with an expression vector comprising an expression cassette further comprising at least one polynucleotide or variant or fragment thereof of the invention. The terms ‘identical’ or percent ‘identity’ in the context of two or more nucleic acids or polypeptide sequences, refer to two or more sequences or subsequences that are the same or have a specified percentage of amino acid residues or nucleotides that are the same (i.e. 70% identity over a specified region), when compared and aligned for maximum correspondence over a comparison window, or designated region as measured using sequence comparison algorithms or by manual alignment and visual inspection. Preferably, the identity exists over a region that is at least about 25 amino acids or nucleotides in length, or more preferably over a region that is 50-100 amino acids or nucleotides or even more in length. Examples of useful algorithms are PILEUP (Higgins & Sharp, CABIOS 5:151 (1989), BLAST and BLAST 2.0 (Altschul et al. J. Mol. Biol. 215: 403 (1990). Software for performing BLAST analyses is publicly available through the National Center for Biotechnology Information (http://www/ncbi.nlm.nih.gov/). In the present invention the term ‘homologue’ also refers to ‘identity’. For example a homologue of a polynucleotide comprising SEQ ID NO: 1-66 has at least 70% identity to one of these sequences. In yet another embodiment such a homologue has at least 75%, 80%, 85% or 90% homology. According to still further features in the described preferred embodiments the polynucleotide fragment encodes a polypeptide able to enhance the production of tabersonine and/or catharanthine and/or vinblastine and/or vincristine, which may therefore be allelic, species and/or induced variant of the amino acid sequence encoded by a sequence comprising SEQ ID NO: 1-66. A variant of a polynucleotide as described herein may be a naturally occurring variant such as an allelic variant, or it may be a variant that is not known to occur naturally. Changes in the nucleotide sequence of the variant may be silent, i.e. they may not alter the amino acids encoded by the polynucleotide. Where alterations are limited to silent changes of this type a variant will encode a polypeptide with the same amino acid sequence. Changes in the nucleotide sequence of the variant may alter the amino acid sequence of the polypeptides encoded by the polynucleotides described herein. Such nucleotide changes may result in amino acid substitutions, additions, deletions, fusions and truncations in the polypeptide encoded by the polynucleotide sequences. Non-naturally occurring variants of polynucleotides and polypeptides may be made by mutagenesis techniques or by direct synthesis. For instance, a conservative amino acid substitution may be made with respect to the amino acid sequence encoding the polypeptide. A “conservative amino acid substitution”, as used herein, is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Nucleic acid molecules corresponding to natural allelic variants and homologues can be isolated based on their homology to the polynucleotides comprising SEQ ID NO: 1-66 disclosed herein using said polynucleotide sequences, or a portion thereof, as a hybridisation probe according to standard hybridisation techniques (e.g. as described in Sambrook, J. et al. molecular Cloning: A Laboratory Manual. 2^{nd} ed., Cold Spring Harbor Laboratory, Cold Spring Harbor, N.Y., 1989).

The present invention accordingly provides in another embodiment a method for enhancing the production of tabersonine and/or catharanthine and/or vinblastine and/or vincristine in plant or plant cells, by transformation of said plant or plant cells with an expression vector comprising an expression cassette that further comprises at least one gene comprising a fragment, variant or homologue encoded by at least one sequence selected from a polynucleotide comprising SEQ ID NO: 1-66.

In another embodiment the invention provides a recombinant DNA vector comprising at least one polynucleotide sequence, homologue, fragment or variant selected from at least one of the sequences comprising SEQ ID NO: 1-66. The vector may be of any suitable type including, but not limited to, a phage, virus, plasmid, phagemid, cosmid, bacmid or even an artificial chromosome. The at least one polynucleotide sequence preferably codes for at least one polypeptide that is involved in the biosynthesis and/or regulation of synthesis of tabersonine and/or catharanthine and/or vinblastine and/or vincristine (e.g. a transcription factor, a repressor, an enzyme that regulates a feed-back loop, a transporter, a chaperone). The term “recombinant DNA vector” as used herein refers to DNA sequences containing a desired coding sequence and appropriate DNA sequences necessary for the expression of the operably linked coding polynucleotide sequence in a particular host organism (e.g. plant cell). Plant cells are known to utilize promoters, polyadenylation signals and enhancers.

In yet another embodiment the invention provides a transgenic plant or derived cell thereof transformed with said recombinant DNA vector.

A recombinant DNA vector comprises at least one “expression cassette”. Expression cassettes are generally DNA constructs preferably including (5′ to 3′ in the direction of transcription): a promoter region, a polynucleotide sequence, homologue, variant or fragment thereof of the present invention operatively linked with the transcription initiation region, and a termination sequence including a stop signal for RNA polymerase and a polyadenylation signal. It is understood that all of these regions should be capable of operating in biological cells, such as plant cells, to be transformed. The promoter region comprising the transcription initiation region, which preferably includes the RNA polymerase binding site, and the polyadenylation signal may be native to the biological cell to be transformed or may be derived from an alternative source, where the region is functional in the biological cell.

The polynucleotide sequence, homologue, variant or fragment thereof of the invention may be expressed—alone or in combination—in for example a plant cell under the control of a promoter that directs constitutive expression or regulated expression. Regulated expression comprises temporally or spatially regulated expression and any other form of inducible or repressible expression. Temporally means that the expression is induced at a certain time point, for instance, when a certain growth rate of the plant cell culture is obtained (e.g. the promoter is induced only in the stationary phase or at a certain stage of development). Spatially means that the promoter is only active in specific organs, tissues, or cells (e.g. only in roots, leaves, epidermis, guard cells or the like). Other examples of regulated expression comprise promoters whose activity is induced or repressed by adding chemical or physical stimuli to the plant cell. In a preferred embodiment the expression is under control of environmental, hormonal, chemical, and/or developmental signals. Such promoters for plant cells include promoters that are regulated by (1) heat, (2) light, (3) hormones, such as abscisic acid and methyl jasmonate (4) wounding or (5) chemicals such as salicylic acid, chitosans or metals. Indeed, it is well known that the expression of secondary metabolites (such as terpenoid indole alkaloids) can be boosted by the addition of for example specific chemicals, jasmonate and elicitors. In a particular embodiment the co-expression of several (more than one) polynucleotide sequence or homologue or variant or fragment thereof, in combination with the induction of terpenoid indole alkaloid synthesis is beneficial for an optimal and enhanced production of tabersonine, catharanthine, vinblastine or vincristine. Alternatively, the at least one polynucleotide sequence comprising SEQ ID NO: 1-66, or a homologue, an variant or a functional fragment thereof is placed under the control of a constitutive promoter. A constitutive promoter directs expression in a wide range of cells under a wide range of conditions. Examples of constitutive plant promoters useful for expressing heterologous polypeptides in plant cells include, but are not limited to, the cauliflower mosaic virus (CaMV) 35S promoter, which confers constitutive, high-level expression in most plant tissues including monocots; the nopaline synthase promoter and the octopine synthase promoter. The expression cassette is usually provided in a DNA or RNA construct which is typically called an “expression vector” which is any genetic element, e.g., a plasmid, a chromosome, a virus, behaving either as an autonomous unit of polynucleotide replication within a cell (i.e. capable of replication under its own control) or being rendered capable of replication by insertion into a host cell chromosome, having attached to it another polynucleotide segment, so as to bring about the replication and/or expression of the attached segment. Suitable vectors include, but are not limited to, plasmids, bacteriophages, cosmids, plant viruses and artificial chromosomes. The expression cassette may be provided in a DNA construct which also has at least one replication system. In addition to the replication system, there will frequently be at least one marker present, which may be useful in one or more hosts, or different markers for individual hosts. The markers may a) code for protection against a biocide, such as antibiotics, toxins, heavy metals, certain sugars or the like; b) provide complementation, by imparting prototrophy to an auxotrophic host: or c) provide a visible phenotype through the production of a novel compound in the plant. Exemplary genes which may be employed include neomycin phosphotransferase (NPTII), hygromycin phosphotransferase (HPT), chloramphenicol acetyltransferase (CAT), nitrilase, and the gentamicin resistance gene. For plant host selection, non-limiting examples of suitable markers are β-glucuronidase, providing indigo production, luciferase, providing visible light production, Green Fluorescent Protein and variants thereof, NPTII, providing kanamycin resistance or G418 resistance, HPT, providing hygromycin resistance, and the mutated aroA gene, providing glyphosate resistance.

The term “promoter activity” refers to the extent of transcription of a polynucleotide sequence, homologue, variant or fragment thereof that is operably linked to the promoter whose promoter activity is being measured. The promoter activity may be measured directly by measuring the amount of RNA transcript produced, for example by Northern blot or indirectly by measuring the product coded for by the RNA transcript, such as when a reporter gene is linked to the promoter. The term “operably linked” refers to linkage of a DNA segment to another DNA segment in such a way as to allow the segments to function in their intended manners. A DNA sequence encoding a gene product is operably linked to a regulatory sequence when it is ligated to the regulatory sequence, such as, for example a promoter, in a manner which allows modulation of transcription of the DNA sequence, directly or indirectly. For example, a DNA sequence is operably linked to a promoter when it is ligated to the promoter downstream with respect to the transcription initiation site of the promoter and allows transcription elongation to proceed through the DNA sequence. A DNA for a signal sequence is operably linked to DNA coding for a polypeptide if it is expressed as a pre-protein that participates in the transport of the polypeptide. Linkage of DNA sequences to regulatory sequences is typically accomplished by ligation at suitable restriction sites or adapters or linkers inserted in lieu thereof using restriction endonucleases known to one of skill in the art.

In a particular embodiment the polynucleotides or homologues or variants or fragments thereof of the present invention can be introduced in plants or plant cells that are different from Catharanthus roseus and said polynucleotides can be used for the enhancement of terpenoid indole alkaloids in plants or plant cells which are different from Catharanthus roseus. Preferred plants (or plant cell lines derived thereof) are plants that produce the secondary metabolite strictosidine. Examples of such plants comprise Vinca major, Qinchona officinalis, Corynanthe johimbe, Strychnos nux-vomica and Rauwolfia serpentine.

The term “heterologous DNA” and or “heterologous RNA” refers to DNA or RNA that does not occur naturally as part of the genome or DNA or RNA sequence in which it is present, or that is found in a cell or location in the genome or DNA or RNA sequence that differs from that which is found in nature. Heterologous DNA and RNA (in contrast to homologous DNA and RNA) are not endogenous to the cell into which it is introduced, but has been obtained from another cell or synthetically or recombinantly produced. An example is a gene isolated from one plant species operably linked to a promoter isolated from another plant species. Generally, though not necessarily, such DNA encodes RNA and proteins that are not normally produced by the cell in which the DNA is transcribed or expressed. Similarly exogenous RNA encodes for proteins not normally expressed in the cell in which the exogenous RNA is present. Heterologous DNA or RNA may also refer to as foreign DNA or RNA. Any DNA or RNA that one of skill in the art would recognize as heterologous or foreign to the cell in which it is expressed is herein encompassed by the term heterologous DNA or heterologous RNA. Examples of heterologous DNA include, but are not limited to, DNA that encodes proteins, polypeptides, receptors, reporter genes, transcriptional and translational regulatory sequences, selectable or traceable marker proteins, such as a protein that confers drug resistance, RNA including mRNA and antisense RNA and ribozymes.

Accordingly, the invention provides in a further aspect a gene construct in the form of an expression cassette comprising as operably linked components in the 5′-3′ direction of transcription, one or more units each comprising a suitable promoter in a plant cell, a plurality of nucleotide sequences selected from the group comprising sequences SEQ ID NO: 1-66 for the stimulation of tabersonine and/or catharanthine and/or vinblastine and/or vincristine production and a suitable transcriptional and translational termination regulatory region. The promoter and termination regulatory regions will be functional in the host plant cell and may be heterologous or homologous to the plant cell and the gene. Suitable promoters which may be used are described above.

The termination regulatory region may be derived from the 3′ region of the gene from which the promoter was obtained or from another gene. Suitable termination regions, which may be used, are well known in the art and include Agrobacterium tumefaciens nopaline synthase terminator (Tnos), Agrobacterium tumefaciens mannopine synthase terminator (Tmas), the rubisco small subunit terminator (TrbcS) and the Ca 35S terminator (T35S).

The present invention can be practiced with any plant variety of Catharanthus roseus or plants that produce terpenoid indole alkaloids for which cells of the plant can be transformed with an expression cassette of the current invention and for which transformed cells can be cultured in vitro. Suspension culture, callus culture, hairy root culture, shoot culture or other conventional plant cell culture methods may be used (as described in: Drugs of Natural Origin, G. Samuelsson, 1999, ISBN 9186174813).

By “plant cells” it is understood any cell which is derived from a plant and can be subsequently propagated as callus, plant cells in suspension, organized tissue and organs (e.g. hairy roots). Tissue cultures derived from the plant tissue of interest can be established. Methods for establishing and maintaining plant tissue cultures are well known in the art (see, e.g. Trigiano R. N. and Gray D. J. (1999), “Plant Tissue Culture Concepts and Laboratory Exercises”, ISBN: 0-8493-2029-1; Herman E. B. (2000), “Regeneration and Micropropagation: Techniques, Systems and Media 1997-1999”, Agricell Report). Typically, the plant material is surface-sterilized prior to introducing it to the culture medium. Any conventional sterilization technique, such as chlorinated bleach treatment can be used. In addition, antimicrobial agents may be included in the growth medium. Under appropriate conditions plant tissue cells form callus tissue, which may be grown either as solid tissue on solidified medium or as a cell suspension in a liquid medium.

A number of suitable culture media for callus induction and subsequent growth on aqueous or solidified media are known. Exemplary media include standard growth media, many of which are commercially available (e.g., Sigma Chemical Co., St. Louis, Mo.). Examples include Schenk-Hildebrandt (SH) medium, Linsmaier-Skoog (LS) medium, Murashige and Skoog (MS) medium, Gamborg's B5 medium, Nitsch & Nitsch medium, White's medium, and other variations and supplements well known to those of skill in the art (see, e.g., Plant Cell Culture, Dixon, ed. IRL Press, Ltd. Oxford (1985) and George et al., Plant Culture Media, Vol 1, Formulations and Uses Exegetics Ltd. Wilts, UK, (1987)). For the growth of conifer cells, particularly suitable media include ½ MS, ½ L.P., DCR, Woody Plant Medium (WPM), Gamborg's B5 and its modifications, DV (Durzan and Ventimiglia, In Vitro Cell Dev. Biol. 30:219-227 (1994)), SH, and White's medium.

In a particular embodiment the current invention can be combined with other known methods to enhance the production and/or the secretion of tabersonine and/or catharanthine and/or vincristine and/or vinblastine in plant cell cultures such as (1) by improvement of the plant cell culture conditions, (2) by the transformation of the plant cells with a transcription factor capable of upregulating genes involved in the pathway of terpenoid indole alkaloid formation, (3) by the addition of specific elicitors to the plant cell culture, and 4) by the induction of organogenesis. The term “plant” as used herein refers to vascular plants (e.g. gymnosperms and angiosperms). The method comprises transforming a plant cell with an expression cassette of the present invention and regenerating such plant cell into a transgenic plant. Such plants can be propagated vegetatively or reproductively. The transforming step may be carried out by any suitable means, including by Agrobacterium-mediated transformation and non-Agrobacterium-mediated transformation, as discussed in detail below. Plants can be regenerated from the transformed cell (or cells) by techniques known to those skilled in the art. Where chimeric plants are produced by the process, plants in which all cells are transformed may be regenerated from chimeric plants having transformed germ cells, as is known in the art. Methods that can be used to transform plant cells or tissue with expression vectors of the present invention include both Agrobacterium and non-Agrobacterium vectors. Agrobacterium-mediated gene transfer exploits the natural ability of Agrobacterium tumefaciens to transfer DNA into plant chromosomes and is described in detail in Gheysen, G., Angenon, G. and Van Montagu, M. 1998. Agrobacterium-mediated plant transformation: a scientifically intriguing story with significant applications. In K. Lindsey (Ed.), Transgenic Plant Research. Harwood Academic Publishers, Amsterdam, pp. 1-33 and in Stafford, H. A. (2000) Botanical Review 66: 99-118. A second group of transformation methods is the non-Agrobacterium mediated transformation and these methods are known as direct gene transfer methods. An overview is brought by Barcelo, P. and Lazzeri, P. A. (1998) Direct gene transfer: chemical, electrical and physical methods. In K. Lindsey (Ed.), Transgenic Plant Research, Harwood Academic Publishers, Amsterdam, pp. 35-55. Hairy root cultures can be obtained by transformation with virulent strains of Agrobacterium rhizogenes, and they can produce high contents of secondary metabolites characteristic to the mother plant. Protocols used for establishing of hairy root cultures vary, as well as the susceptibility of plant species to infection by Agrobacterium (Toivonen L. (1993) Biotechnol. Prog. 9, 12; Vanhala L. et al. (1995) Plant Cell Rep. 14, 236). It is known that the Agrobacterium strain used for transformation has a great influence on root morphology and the degree of secondary metabolite accumulation in hairy root cultures. It is possible that by systematic clone selection e.g. via protoplasts, to find high yielding, stable, and from single cell derived-hairy root clones. This is possible because the hairy root cultures possess a great somaclonal variation. Another possibility of transformation is the use of viral vectors (Turpen T H (1999) Philos Trans R Soc Lond B Biol Sci 354(1383): 665-73).

Any plant tissue or plant cells capable of subsequent clonal propagation, whether by organogenesis or embryogenesis, may be transformed with an expression vector of the present invention. The term ‘organogenesis’ means a process by which shoots and roots are developed sequentially from meristematic centers; the term ‘embryogenesis’ means a process by which shoots and roots develop together in a concerted fashion (not sequentially), whether from somatic cells or gametes. The particular tissue chosen will vary depending on the clonal propagation systems available for, and best suited to, the particular species being transformed. Exemplary tissue targets include protoplasts, leaf disks, pollen, embryos, cotyledons, hypocotyls, megagametophytes, callus tissue, existing meristematic tissue (e.g. apical meristems, axillary buds, and root meristems), and induced meristem tissue (e.g., cotyledon meristem and hypocotyls meristem).

In yet another embodiment suitable expression cassettes comprising the nucleotide sequences of the present invention can be used for transformation into other species (different from Catharanthus roseus). This transformation into other species or genera can be carried out randomly or can be carried out with strategically chosen nucleotide sequences. The random combination of genetic material from one or more species of organisms can lead to the generation of novel metabolic pathways (for example through the interaction with metabolic pathways resident in the host organism or alternatively silent metabolic pathways can be unmasked) and eventually lead to the production of novel classes of compounds. This novel or reconstituted metabolic pathways can have utility in the commercial production of novel, valuable terpenoid indole alkaloids.

Various assays within the knowledge of the person skilled in the art may be used to determine whether the plant cell shows an increase in gene expression, for example, Northern blotting or quantitative reverse transcriptase PCR (RT-PCR). Whole transgenic plants may be regenerated from the transformed cell by conventional methods. Such transgenic plants having improved terpenoid indole alkaloid (e.g. tabersonine, catharanthine, vinblastine or vincristine) levels may be propagated and crossed to produce homozygous lines. Such plants produce seeds containing the genes for the introduced trait and can be grown to produce plants that will produce the selected phenotype.

The recombinant DNA and molecular cloning techniques applied in the below examples are all standard methods well known in the art and are e.g. described by Sambrook et al. (1989) Molecular cloning: A laboratory manual, second edition, Cold Spring Harbor Laboratory Press. Methods for tobacco cell culture and manipulation applied in the below examples are methods described in or derived from methods described in Nagata et al. (1992) Int. Rev. Cytol. 132, 1.

### EXAMPLES

### 1. Growth and Targeted TIA Analysis

In order to enable functional genomics driven gene discovery in TIA metabolism in Madagascar periwinkle cells, it is crucial to establish the conditions in which differential accumulation of the desired metabolites can be observed. This can for instance be achieved by studying different plant developmental stages or the effects of elicitor treatments. In the present invention it is assumed that developmental or environmental conditions affecting the production of the desired secondary metabolites will also activate the genes involved in the biosynthesis of such compounds (Oksman-Caldentey and Inzé, 2004). In the case of C. roseus, literature suggests that TIA accumulation is strongly influenced by the complex interaction of phytohormones such as auxins and jasmonates (Gantet et al., 1998; Pasquali et al., 1992). Therefore, it seemed most promising to investigate the combined effects of these two hormones on TIA accumulation in C. roseus cells, applying similar growth and elicitation conditions as described (Gantet et al., 1998). Since alkaloid contents can vary greatly between different cell lines, two independent C. roseus cell lines were used in this study, namely ‘Leiden’ and ‘Wuerzburg’ (see Experimental procedures). Growth curves for both cell lines and two experimental conditions (i.e. with and without auxin) were determined to establish the optimal time for elicitation. The elicitation point for both cell lines was set at day six. At that time cultured cells of both strains, under all growth conditions (with and without auxin) were in middle or late growth phase. In contrast to the ‘Wuerzburg’ line, the ‘Leiden’ line has been specifically optimized for TIA production. This was clearly reflected by higher absolute TIA levels and a more pronounced response to MeJA elicitation in the ‘Leiden’ cells. Consequently, in this invention we have concentrated on the ‘Leiden’ line for further profiling analyses. Tabersonine levels in ‘Leiden’ cells grown in the presence of auxin showed an extremely fast and pronounced response. Already 4 h after elicitation they had accumulated from 13±1 to 124±5 μg/g dw and after 12 h to 141±7 μg/g dw representing a more than 10 fold increase. In the absence of auxin, elicitation was observed as well, although to a lesser extent (from <1 to 5 μg/g dw). In general, catharanthine was present at levels reported in the literature (Vazquez-Flota et al., 2002) in the ‘Leiden’ line. When jasmonate elicited cells were grown in the presence of auxin, catharanthine accumulated up to 360±69 μg/g dw, a level that had been reached with other elicitors, too (Zhao et al., 2001). In contrast, in cells cultured without auxin a differential elicitation effect could not be observed with respect to catharanthine concentrations. Similarly, ajmalicine concentrations were in the same range as reported in the literature (Vazquez-Flota et al., 2002). The amounts after elicitation approximately doubled within 12 h in medium supplemented with auxin, whereas in the absence of auxin jasmonate-mediated induction of ajmalicine accumulation seemed to occur with some delay. Nonetheless, in accordance with Gantet et al. (1998) removal of auxins by itself, even in the absence of MeJA, positively affected TIA biosynthesis, leading to similar accumulation levels as obtained with MeJA elicitation in the presence of auxins. Notably, in ‘Leiden’ cells catharanthine and tabersonine levels are positively affected by the presence of auxins, whereas the opposite is true for ajmalicine accumulation, pointing to a possible divergent regulation of different steps of the TIA biosynthetic pathway by phytohormones. The negative effect of auxins on ajmalicine levels has been also observed by Gantet et al. (1998), whereas levels of other TIAs were not assessed in the latter study. In striking contrast however, ajmalicine levels in ‘Wuerzburg’ cells are positively affected by the presence of auxins, underscoring the existence of variability in the regulation of TIA biosynthesis in different cell lines.

As expected (St-Pierre et al., 1999) vindoline and the downstream bisindolalkaloids vinblastine and vincristine were not detected in quantifiable amounts in none of the cell lines under any of the conditions tested.

### 2. Quantitative Evaluation of cDNA-AFLP Profiling of C. roseus Gene Expression

Using 128 BstYI+1/MseI+2 primer combinations, quantitative temporal accumulation patterns of 10,790 transcript tags were determined and analyzed from the ‘Leiden’ C. roseus cell culture. In total, 561 differentially expressed transcript tags were isolated. Direct sequencing of the PCR products gave good-quality sequences for 417 (=74%) fragments. To the remaining 26%, no unique sequence could be attributed unambiguously, indicating that they might not represent unique gene tags. Similar performance numbers were obtained in the cDNA-AFLP based transcript profiling of methyl jasmonate elicited tobacco BY-2 cells (Goossens et al., 2003). Homology searches with the sequences from the unique C. roseus (CR) gene tags revealed that 63% of these tags displayed similarity with known plant genes, whereas for the remaining 37% of the tags no homology to a known sequence was found. When blasting the nucleotide sequences of the CR cDNA-AFLP tags with the 236 prior to this study publicly available C. roseus EMBL entries, only 33 hits were found with a (near) 100% match. The majority of the tags identified in this study thus represent novel gene sequences of the medicinal plant C. roseus.

### 3. Qualitative Evaluation of cDNA-AFLP Profiling of C. roseus Gene Expression

The CR tags can be divided in different subclusters, either based on their expression profiles or based on their annotation. Average linkage hierarchical clustering analysis of the expression profiles showed that three main forces steer the formation of clusters. Listed by their order of impact, these are the addition of methyl jasmonate, growth (independent of the exogenous application of hormones or elicitors), and the presence of auxin, modulating respectively the expression of 42.8%, 33.8%, and 28.5% of the CR tags. For 5.2% of the CR tags the expression is affected both by methyl jasmonate and auxin. According to the Functional Catalogue of the Munich Information Center for Protein Sequences (http://mips.gsf.de/projects/funcat) the CR tags could be classified into eight broadly defined functional groups. Notably, and of relevance for this study is that the functional category group ‘Metabolism & Energy’ constitutes one of the major groups, including as anticipated a large number of TIA genes, but also a notable number of tags with sequence similarity to genes encoding enzymes involved in the S-adenosylmethionine (SAM) cycle, phytohormone metabolism, and phenolic compound biosynthesis. To assess the relevance of the cDNA-AFLP tags presented in this study, a biological repeat experiment was performed on the ‘Wuerzburg’ line submitted to the same treatments. 18 primer combinations were chosen and run on the ‘Wuerzburg’ samples (biological repeat) and again on the ‘Leiden’ samples (technical repeat). In the ‘Leiden’ CR tag collection, 70 unique differentially expressed tags were isolated using these particular primer combinations. The technical repeat confirmed the expression profile for more than 90% of the CR tags whereas the biological repeat showed that 57% of the tags differentially regulated in the ‘Leiden’ culture have a gene equivalent in the ‘Wuerzburg’ culture that displays a similar expression pattern. When excluding CR tags that subcluster in the ‘Growth regulated’ groups, which likely represent genes not directly related to TIA biosynthesis, the biological repeat gives a higher match (63%). One should also consider that nuclear polymorphisms between the two cell lines (and thus the C. roseus cultivars they were derived off) might account in part for the differential AFLP patterns. Based on this partial cDNA-AFLP analysis it could be estimated that overall 3% of the transcript tags show a cell line specific presence.

### 4. CR Tags Corresponding to Genes Involved in TIA Biosynthesis

The tags corresponding to genes reported to be associated with TIA biosynthesis that were isolated as differentially expressed by cDNA-AFLP include tags corresponding with genes associated with the biosynthesis of the terpenoid moiety (1-deoxy-D-xylulose-5-phosphate synthase, DXS; 2C-methyl-D-erythrol-2,4-cyclodiphosphate synthase, MECS, 1-hydroxy-2-methyl-2-(E)-butenyl 4-diphosphate synthase, HDS, geranylpyrophosphate synthase, GPPS and geraniol 10-hydroxylase, G10H), biosynthesis of the indole moiety (anthranilate synthase, AS), biosynthesis of the monomer TIAs (strictosidine β-D-glucosidase, SGD) and transcription factors regulating TIA biosynthesis (octadecanoid-responsive Catharanthus AP2-domain factors, ORCA2 and ORCA3). In all these cases, it concerns tags with a (near) perfect match with the gene sequences, encoding the isoforms reported to catalyze (or regulate) the known enzymatic reactions. Additionally, the cDNA-AFLP set also includes tags with close similarity (greater than 70%) to 10-hydroxygeraniol oxidoreductase (10HGO), strictosidine synthase (STR), tabersonine 16-hydroxylase (T16H) and deacetylvindoline 4-O-acetyltransferase (DAT) but of which the sequence does not perfectly match the isoenzyme reported to catalyze the corresponding enzymatic reactions. Further analysis will be required to investigate whether the gene products corresponding to these tags catalyze identical reactions or whether they possibly generate structurally related alkaloids or other types of secondary metabolites. Expression of all these genes is induced by elicitation with MeJA, and additionally either stimulated or repressed by the presence of auxin. RT-PCR was performed for all of the remaining known but undetected TIA biosynthesis genes and showed that all but two of them (i.e. T16H and tryptophan decarboxylase, TDC) either were not differentially expressed or not transcribed at all. The expression profiles (snapshot at 12 h after elicitation) of the TIA genes are also displayed together with the targeted TIA metabolite profiling data in the pathway presented in FIG. 1.

### 5. Overlap Between Transcriptome and Proteome Analysis

Recently, a systematic 2D-PAGE proteomic analysis on TIA producing C. roseus cell suspension cultures at various points in a culture growth cycle allowed the identification of 58 C. roseus proteins, including tryptophan synthase and two isoforms of STR (Jacobs et al., 2005). By comparing the sequences of this proteome data set with our transcriptome sequence data set, for nine of the protein spots, a potential corresponding transcript tag could be identified. By BlastX searches of the transcript tag nucleotide sequences against the protein spot amino acid sequences, a direct hit (with 88% to 100% identity) was found with three protein spots, amongst which the so-called PS Protein, suggested to be associated with TIA biosynthesis (Lemenager et al., 2005). By looking for common hits of respective Blast searches with the transcript tag and protein spot sequences against public databases, presumable counterparts for six more protein spots could be detected within the transcript tag set, amongst which was an enzyme involved in jasmonate biosynthesis (i.e. 12-oxophytodienoate reductase). As expected a common blast hit was also encountered with the STR-like transcript tag (CRG130) and the two STR isoforms identified by Jacobs et al. (2005), the latter displaying a 100% match with the previously released STR gene sequence (McKnight et al., 1990), in contrast to the CRG130 sequence.

### 6. Non-Targeted Metabolite Analysis

To allow true integration of metabolomics and transcriptomics and ultimately collect novel information on gene-to-metabolite networks in C. roseus cells, we performed a large-scale non-targeted LC-MS analysis of the same samples as used for cDNA-AFLP profiling. Prior to normalization and peak filtering the metabolic profile of this non-targeted analysis revealed 3891 peaks using the MZmine LC/MS toolbox (Katajamaa and Oresis, 2005). Among those there were many peaks with too low signal-to-noise ratio to be considered relevant to the study, such as peaks of very low intensity and low variability, and natural isotope peaks. We have therefore performed peak filtering to remove such peaks. Furthermore, we have excluded the peaks eluting within the first three minutes, because retention is unstable within this period. The peak filtering resulted in 178 peaks altogether. Using an internal library of masses and retention times of metabolites from the TIA biosynthetic pathway, we identified nine compounds (ajmalicine, tabersonine, catharanthine, yohimbine, cathenamine, secologanine, lochnerinine, 16-methoxy-2,3-dihydro-3-hydroxytabersonine, and desacetoxyvindoline). As anticipated all of these compounds are synthesized upstream of D4H in the pathway (FIG. 1B). The remaining peaks comprised metabolites most abundantly in the range of 300-400 m/z, the typical range expected for the monomeric TIA metabolite class, some of them also displaying retention times similar to the identified TIAs. Notwithstanding, more detailed analytical investigation would be required for unequivocal chemical characterisation. Average linkage hierarchical clustering of the metabolite accumulation profiles reveals the existence of various subclusters of metabolites, in which two are of particular importance for this study: one consisting of 43 metabolites of which accumulation is stimulated by auxins and one consisting of 53 metabolites of which accumulation is repressed by auxins. To the best of our knowledge, this is the first report that provides a list, obtained through non-targeted metabolite profiling and consisting of known and novel metabolites of which accumulation is modulated by an auxin. Eventually, for some of the metabolites present in these two clusters, the differential auxin-mediated regulation might be enforced by or depends on the presence of MeJA. Most important however is the fact that not all identified monomeric TIAs group within the same auxin-modulated subcluster. For instance, the accumulation of tabersonine and catharanthine, the two building blocks for bisindole alkaloid biosynthesis, is positively affected by auxins whereas the accumulation of ajmalicine, a monomeric TIA not involved in the synthesis of bisindole alkaloids, is repressed by the presence of auxins. This was apparent from the targeted TIA analysis, too, and similar evidence was also obtained from the Principal Components Analysis and linear network correlation analysis (see below). This indicates the existence of divergent regulation of biosynthesis and accumulation of metabolites within the TIA pathway.

### 7. Integrated Analysis of Transcriptome and Metabolome

For integrated analysis of metabolite and transcript profiles, the accumulation profiles of 178 retained metabolite peaks were combined with the expression profiles of the 471 transcripts. Normalized profiling data were scaled so that the mean value within each of the two datasets was set to 1. In order to explore the variability structure of data, we first performed the Principal Components Analysis (PCA) using mean scaling (Jackson, 1991). The first two principal components (PC), accounting for 64% of total variability, reveal clear separation between the auxin-treated and non-treated cells by first PC, and the separation of each group across the time domain along the second PC. To establish gene-to-gene and metabolite-to-gene co-regulation patterns, we utilized correlation network analysis (Kose et al., 2001; Oresis et al., 2004). We calculated the Pearson correlation coefficient between each pair of variables (either gene or metabolite) across the profiles including all time points and conditions. To optimally visualise the complex networks related to secondary metabolism in C. roseus cells, we performed the analysis for two different subsets of the profiles. As illustrated below these two approaches clearly underscore the power of integrative metabolite and transcript profiling. Out of the hundreds of metabolite peaks and transcript tags identified one can sort the ones of which accumulation or expression is closely correlated with the accumulation of certain TIA metabolites. In first instance, correlation network analysis was performed for a selected subset of identified metabolites and genes. This subset was chosen arbitrarily and consisted at the one hand of the nine identified TIA metabolite peaks and at the other hand of the gene tags identical to or with close sequence similarity to genes respectively encoding proteins catalysing jasmonate and TIA biosynthesis, and given the importance of these two protein families in TIA biosynthesis, all putative cytochrome P450 (CP450) and AP-2 transcription factors. All of the CR tags belonging to the gene classes used in this subset could be integrated in the network, except for three tags corresponding respectively to an AP-2 factor (CRG20), a lipoxygenase possibly involved in jasmonate biosynthesis (CRG48), and a CP450 (CRG96). Most striking observations here are (i) the presence of a strongly correlated gene-to-gene network that includes practically all of the known early pathway TIA genes as well as the ORCA transcription factors, (ii) the presence of a gene-to-metabolite cluster comprising a part of the TIA metabolites and a set of CP450 genes with still unspecified function, and (iii) some smaller correlation groups (i.e. only pairs or triplets of genes or metabolites), containing for instance ajmalicine, thus confirming the differential regulation of this particular biosynthetic branch. Clearly, the correlation network analysis conveniently allows distinguishing within the genes of unknown function (for instance putative CP450s) the ones that are most interesting by linking some of them with particular branches of the TIA biosynthetic pathway. Secondly, an unbiased subset is visualised, subtracted from the complete network across all transcript and metabolite profiles, and centring on the tabersonine node. The gene-to-metabolite network around this node consists of 29 metabolites and 97 genes (with a blast hit) that represent the nearest links). Importantly, many of the unassigned metabolites that are included in this cluster display masses in the range of 300-400 m/z, the typical range expected for the monomeric TIA metabolite class, and thus might represent as yet unknown intermediates or side products of TIA metabolism. Accordingly, this network centred on tabersonine harbours 66 tags corresponding to enzymes with still unknown activity and substrate specificity, thus candidates to code for some of the missing links in the biosynthesis of tabersonine and/or catharantine and/or vinblastine and/or vincristine.

### 8. Functional Validation of the Polynucleotides Comprising SEQ ID NO: 1-66 in Transgenic Hairy Roots of Catharanthus roseus

For the cloning of the complete genes, corresponding to the gene tags with SEQ ID NO:1-66, full-length open reading frames (ORFs) are isolated either via RACE technology (Invitrogen) or by screening of a Catharanthus roseus cDNA library. This custom library (Invitrogen) was derived from Catharanthus roseus cells harvested at different time points following MeJA elicitation. Library screening of this library is performed either via colony hybridization using cDNA-AFLP tag sequences as probes or via PCR. For RACE and PCR screening, primers were designed based on available cDNA-AFLP tag sequences using the Primer3 software (Rozen and Skaletsky, 2000). Full-length ORF amplicons are synthesized by PCR using the Platinum Pfx DNA polymerase (Invitrogen) with the Gateway attB1 and attB2 sequences, and are subsequently transferred, using the Gateway recombination cloning technology (Invitrogen), in pDONR221 via BP clonase, then the binary vector pK7WG2D via LR clonase (Karimi et al., 2002). Each ORF is fully sequence validated in the pDONR221 entry clone. The pK7WG2D constructs are then finally transferred to an Agrobacterium rhizogenes strain suitable for Catharanthus roseus hairy root transformation.

The generation of hairy roots is as follows. Fully expanded green leaves of greenhouse-grown C. roseus plants are cut at the base near the stem and surface sterilized by a quick dip in 70% ethanol followed by immersion in 0.6% hypochlorite solution containing 2 drops of tween 20 for 10 min on a magnetic stirrer. Then the leaves are washed two times for 5 min with sterile water and subsequently dried between sterile paper towels. The leaves are placed upside down in petridishes containing MS medium with 10 g/l sucrose. A sterile needle is dipped into the bacterial culture and then used to make incisions in the midrib of the leaves. Cocultivation is for 2 d under light at 23° C. To kill the remaining Agrobacteria the leaves are then transferred to plates containing the same medium but with the addition of 500 mg/l Cefotaxime. Until the appearance of hairy roots the leaves are subcultured every two weeks to the same medium. Finally short pieces of hairy roots are placed on antibiotic free medium.

### 9. Functional Validation of the Polynucleotides Comprising SEQ ID NO: 1-66 in Cell Lines of Catharanthus roseus

The pK7WG2D plasmid constructs generated for hairy root transformation can also be used for transformation of Catharanthus roseus cell lines. To this end the generated constructs are not integrated in an Agrobacterium rhizogenes strain but instead in an Agrobacterium tumefaciens strain suitable for Catharanthus roseus cell line transformation.

Transformation of cells in suspensions is as follows. Catharanthus cells grown as suspension cultures can be transformed either indirect using Agrobacterium tumefaciens or direct by particle bombardment. Indirect transformation has been described by Talou et al. (2001) Plant Phys. and Biochem. 39: 595. based on work of van der Fits (2000) Doctoral Thesis, University of Leiden, Netherlands. Briefly, one volume of bacterial culture is mixed with nine volumes of plant cells and plated on cocultivation medium containing 100 μl acetosyringone. After 3 d incubation in the dark at 24° C. the cells are filtered on sterile filter paper and washed two times with culture medium. For agrokilling and selection the cells are transferred to medium containing cefotaxime and the selective antibiotics.

An efficient direct transformation system using particle bombardment has been described by Hilliou et al. (1999) Plant Science 140: 179. Cells growing on solid medium are dried on sterile filter paper directly before bombardment. 10 mg of 1 μm gold particles coated with 10 μg of DNA are bombarded at 14 kV. Two different plasmids are mixed at a 3:1 molar ratio for cotransformation. The calli are incubated overnight in the dark at 25° C. after the bombardment and are then subcultured to fresh medium. After 5 d the calli are transferred to medium containing the selective antibiotics.

### Materials and Methods

### Plant Cell Cultures and Elicitation

Catharanthus roseus cell suspensions were grown in liquid Gamborg B5 medium (Gamborg et al., 1968) containing 20 g/l sucrose and 1.86 mg/l NM at 26° C., continuous light and 125 rpm shaker speed. Two cell lines designated ‘Leiden’ and ‘Wuerzburg’ were used. The ‘Leiden’ line was a gift from Prof. Robert Verpoorte, Leiden University, and was specifically selected for catharanthine production (Verpoorte, personal communication). The ‘Wuerzburg’ line was established in our laboratory on the same medium and starting from leaves from axenically grown C. roseus seedlings. Growth curves were determined by weighing the lyophilized cells at different time intervals. Elicitations were started at day six after inoculating 2 g fresh weight of cells in 25 ml medium (with or without NM) contained in 100 ml Erlenmeyer flasks by addition of either MeJA dissolved in DMSO at a final concentration of 50 μM or the same volume of DMSO alone as a control. Samples for metabolite and transcript profiling were harvested by vacuum filtration 0, 1, 4, 6, 8, 10 and 12 h after elicitation or 0, 4, 8 and 12 h after DMSO addition. Samples were lyophilized and stored at −20° C. until the extraction was performed.

### Reference Compounds

Pure terpenoid indole alkaloids (Toivonen et al., 1991) ajmalicine hydrochloride, catharanthine hydrochloride, tabersonine hydrochloride and vindoline were generously provided by M. Ojala (VTT Processes, Espoo, Finland). Vinblastine sulfate, vincristine sulfate and vincamine were purchased from Sigma (St. Louis, Mo., USA).

### Extraction and Sample Preparation

For TIA extraction we followed the modified protocol of Whitmer et al. (2002). Briefly, 100 mg of lyophilized cells were spiked with internal standard (vincamine) and extracted with 15 ml ethanol in an ultrasonic bath for 10 min. Following centrifugation at 5000 rpm for 10 min the solvent was decanted and evaporated to dryness. Dry samples were stored at −20° C. until analysis. Then the samples were redissolved in a 1:1 mixture of acetonitrile (Rathburn, Walkerburn, UK) and 10 mM ammoniumacetate (Merck, Darmstadt, Germany) adjusted to pH 10. 25 μl of the solution were injected into the HPLC after centrifugation.

### HPLC-MS Analysis

25 μl sample was loaded onto a reverse-phase C18 column (Xterra MS C18, 4.6×150 mm, 5 μm, Waters) at 35° C. The sample was eluted within 30 min using isocratic conditions of 10 mM ammoniumacetate at pH 10 and acetonitrile (55:45) applying a flow of 1 ml/min and a split of 0.2 ml/min reaching the mass spectrometer. For TIA-targeted analysis HPLC separation was performed using a Waters HT-Alliance 2795 system and was monitored with a Waters 996 photodiode array detector (200 to 270 nm) and a Micromass Quattro micro triple quadrupole mass spectrometer equipped with an electrospray source. The ion source was operated at capillary voltage 3.20 kV and cone voltage 45 V. Source and desolvation temperatures were 130° C. and 290° C., respectively. Desolvation gas flow was 900 l/h and cone gas flow 30 l/h. The selected ion recording (SIR) function was applied to record the protonated molecular ions m/z in 6 channels: 337.1 for catharanthine and tabersonine, 353.1 for ajmalicine, 355.1 for the internal standard vincamine, 457 for vindoline, 811.3 for vinblastine and 825.2 for vincristine. Quantification for ajmalicine, catharanthine, tabersonine, vindoline, vinblastine and vincristine was based on the internal standard method similar to the description by Auriola et al. (1990). Ten-point calibration curves were created (0.004-2 ppm) with five injections each. The amounts of analyte were plotted versus the response. Linear regression was used for calculating the curve parameters. For non-targeted analysis the same Waters HT-Alliance 2795 system with the Micromass Quattro micro triple quadrupole mass spectrometer as for the TIA-targeted analysis was used. All settings were the same except that the full scan mode function was applied. The data processing methods involving peak detection, alignment, and normalization were the same as described previously (Ore\check{s}is et al., 2005).

cDNA-Amplified Fragment Length Polymorphism (AFLP) Analysis and Data Processing

RNA from both ‘Leiden’ and ‘Wuerzburg’ C. roseus cells was prepared with Concert™ Plant RNA Reagent (InVitrogen Life technologies). Sample preparation and cDNA-AFLP based transcript profiling were performed as described (Breyne et al., 2003). All 128 possible BstYI+1/MseI+2 primer combinations were used for transcript profiling on ‘Leiden’ samples, whereas only 18 primer combinations were used on ‘Wuerzburg’ samples. Data processing was performed essentially as described (Vandenabeele et al., 2003). For normalization within each primer combination, 25% of the genes with the lowest coefficient of variation (CV) value were marked as constitutively expressed. Gene tags displaying expression values with a CV>0.6 were considered as differentially expressed and, after visual inspection, were taken for further analysis. To characterize the isolated cDNA-AFLP fragments, the sequences, directly obtained from the reamplified PCR product, were compared against nucleotide and protein sequences in the publicly available databases by BLAST sequence alignments (Altschul et al., 1997). When available, tag sequences were replaced with longer EST or isolated cDNA sequences to increase the chance of finding significant homology. The similarity threshold maintained for BLAST searches was established at a value of 10e-3. However, due to the small size of some tags, in some cases a lower e value was accepted when unambiguous matches were observed.

### Reverse Transcriptase-PCR (RT-PCR)

RNA and single-stranded cDNA for RT-PCR analysis was prepared as described above for cDNA-AFLP analysis. Expression of gene products reported to be involved in TIA biosynthesis was verified by RT-PCR using gene specific primer pairs. PCR-products were visualized on ethidium-bromide stained agarose gels.

### Data Analysis of Transcriptional and Metabolic Profiles

Both metabolic and gene expression datasets were normalized separately with the method based on maximum likelihood estimate of scaling parameters (Hartemink et al., 2001), using the complete dataset in parameter estimation calculations (Oresis et al., 2004). In order to better match the size of the transcriptional dataset, we filtered the peaks from the metabolic profile dataset based on coefficient of variance (CV) and retention time. Only peaks with CV>0.7 across all conditions and with retention time higher than 3 minutes were retained. The Principal Components Analysis (Jackson, 1991) was performed using PLS Toolbox package (Eigenvector Research, Inc.) using Matlab software (MathWorks, Inc.). We used the Tom Sawyer Visualization 6.0 software (Tom Sawyer, Inc.) for generation of correlation networks.

### REFERENCES

Altschul, S. F., Madden, T. L., Schaffer, A. A., Zhang, J. H., Zhang, Z., Miller, W. and Lipman, D. J. (1997) Gapped BLAST and PSI-BLAST: a new generation of protein database search programs. Nucleic Acids Res 25, 3389-3402.
Auriola, S., Naaranlahti, T., Kostiainen, R. and Lapinjoki, S. P. (1990) Determination of Catharanthus alkaloids in plant-cell cultures by thermospray liquid-chromatography mass-spectrometry. Biomed Environ Mass 19, 609-612.
Avdiushko, S., Croft, K. P. C., Brown, G. C., Jackson, D. M., Hamiltonkemp, T. R. and Hildebrand, D. (1995) Effect of volatile methyl jasmonate on the oxylipin pathway in tobacco, cucumber, and Arabidopsis. Plant Physiol 109, 1227-1230.
Breyne, P., Dreesen, R., Cannoot, B., Rombaut, D., Vandepoele, K., Rombauts, S., Vanderhaeghen, R., Inze, D. and Zabeau, M. (2003) Quantitative cDNA-AFLP analysis for genome-wide expression studies. Mol Genet Genomics 269, 173-179.
Chatel, G., Montiel, G., Pre, M., Memelink, J., Thiersault, M., Saint-Pierre, B., Doireau, P. and Gantet, P. (2003) CrMYC1, a Catharanthus roseus elicitor- and jasmonate-responsive bHLH transcription factor that binds the G-box element of the strictosidine synthase gene promoter. J Exp Bot 54, 2587-2588.
Collu, G., Unver, N., Peltenburg-Looman, A. M. G., van der Heijden, R., Verpoorte, R. and Memelink, J. (2001) Geraniol 10-hydroxylase, a cytochrome P450 enzyme involved in terpenoid indole alkaloid biosynthesis. Febs Lett 508, 215-220.
Fontecave, M., Atta, M. and Mulliez, E. (2004) S-adenosylmethionine: nothing goes to waste. Trends Biochem Sci 29, 243-249.
Gamborg, O. L., Miller, R. A. and Ojima, K. (1968) Nutrient requirements of suspension cultures of soybean root cells. Exp. Cell Res. 50, 151-158.
Gantet, P., Imbault, N., Thiersault, M. and Doireau, P. (1998) Necessity of a functional octadecanoic pathway for indole alkaloid synthesis by Catharanthus roseus cell suspensions cultured in an auxin-starved medium. Plant Cell Physiol 39, 220-225.
Goossens, A., Hakkinen, S. T., Laakso, I., Seppanen-Laakso, T., Biondi, S., De Sutter, V., Lammertyn, F., Nuutila, A. M., Soderlund, H., Zabeau, M., Inze, D. and Oksman-Caldentey, K. M. (2003) A functional genomics approach toward the understanding of secondary metabolism in plant cells. P Natl Acad Sci USA 100, 8595-8600.
Hartemink, A. J., Gifford, D. K., Jaakkola, T. S. and Young, R. A. (2001) Maximum likelihood estimation of optimal scaling factors for expression array normalization. In Microarrays: optical technologies and informatics. Proceedings of SPIE (vol. 4266). (Bittner, M., Chen, Y. and Dorsel, A.). 132-140.
Hirai, M. Y., Yano, M., Goodenowe, D. B., Kanaya, S., Kimura, T., Awazuhara, M., Arita, M., Fujiwara, T. and Saito, K. (2004) Integration of transcriptomics and metabolomics for understanding of global responses to nutritional stresses in Arabidopsis thaliana. P Natl Acad Sci USA 101, 10205-10210.
Irmler, S., Schroder, G., St-Pierre, B., Crouch, N. P., Hotze, M., Schmidt, J., Strack, D., Matern, U. and Schroder, J. (2000) Indole alkaloid biosynthesis in Catharanthus roseus: new enzyme activities and identification of cytochrome P450CYP72A1 as secologanin synthase. Plant J 24, 797-804.
Jackson, J. E. (1991) User's guide to principal components New York, N.Y. John Wiley & Sons.
Jacobs, D. I., Gaspari, M., van der Greef, J., van der Heijden, R. and Verpoorte, R. (2005) Proteome analysis of the medicinal plant Catharanthus roseus. Planta 221, 690-704.
Karimi, M., Inzé, D. and Depicker, A. (2002) GATEWAY™ vectors for Agrobacterium-mediated plant transformation. Trends Plant Sci. 7, 193-195.
Katajamaa, M. and Oresic, M. (2005) Processing methods for differential analysis of LC/MS profile data. BMC Bioinformatics 6, 179.
Kose, F., Weckwerth, W., Linke, T. and Fiehn, O. (2001) Visualizing plant metabolomic correlation network using clique-metabolite matrices. Bioinformatics 17, 1198-1208.
Laflamme, P., St-Pierre, B. and De Luca, V. (2001) Molecular and biochemical analysis of a Madagascar periwinkle root-specific minovincinine-19-hydroxy-O-acetyltransferase. Plant Physiol 125, 189-198.
Lemenager, D., Ouelhazi, L., Mahroug, S., Veau, B., St-Pierre, B., Rideau, M., Aguirreolea, J., Burlat, V. and Clastre, M. (2005) Purification, molecular cloning, and cell-specific gene expression of the alkaloid-accumulation associated protein CrPS in Catharanthus roseus. J Exp Bot 56, 1221-1228.
Liechti, R. and Farmer, E. E. (2002) The jasmonate pathway. Science 296, 1649-1650.
McKnight, T. D., Roessner, C. A., Devagupta, R., Scott, A. I. and Nessler, C. L. (1990) Nucleotide-sequence of a cDNA-encoding the vacuolar protein strictosidine synthase from Catharanthus roseus. Nucleic Acids Res 18, 4939-4939.
Memelink, J., Verpoorte, R. and Kijne, J. W. (2001) ORCAnization of jasmonate-responsive gene expression in alkaloid metabolism. Trends Plant Sci 6, 212-219.
Menke, F. L. H., Champion, A., Kijne, J. W. and Memelink, J. (1999) A novel jasmonate- and elicitor-responsive element in the periwinkle secondary metabolite biosynthetic gene Str interacts with a jasmonate- and elicitor-inducible AP2-domain transcription factor, ORCA2. Embo J 18, 4455-4463.
Miyawaki, K., Matsumoto-Kitano, M. and Kakimoto, T. (2004) Expression of cytokinin biosynthetic isopentenyltransferase genes in Arabidopsis: tissue specificity and regulation by auxin, cytokinin, and nitrate. Plant J 37, 128-138.
Moreno, P. R. H., Vanderheijden, R. and Verpoorte, R. (1995) Cell and tissue cultures of Catharanthus roseus—a literature survey. 2. Updating from 1988 to 1993. Plant Cell Tiss Org 42, 1-25.
Nelson, D. R., Koymans, L., Kamataki, T., Stegeman, J. J., Feyereisen, R., Waxman, D. J., Waterman, M. R., Gotoh, O., Coon, M. J., Estabrook, R. W., Gunsalus, I. C. and Nebert, D. W. (1996) P450 superfamily: Update on new sequences, gene mapping, accession numbers and nomenclature. Pharmacogenetics 6, 1-42.
Nikiforova, V. J., Daub, C. O., Hesse, H., Willmitzer, L. and Hoefgen, R. (2005) Integrative gene-metabolite network with implemented causality deciphers informational fluxes of sulphur stress response. J Exp Bot 56, 1887-1896.
Noble, R. L. (1990) The discovery of the Vinca alkaloids—chemotherapeutic agents against cancer. Biochem Cell Bio 168, 1344-1351.
Oksman-Caldentey, K. M. and Inze, D. (2004) Plant cell factories in the post-genomic era: new ways to produce designer secondary metabolites. Trends Plant Sci 9, 433-440.
Oksman-Caldentey, K. M. and Saito, K. (2005) Integrating genomics and metabolomics for engineering plant metabolic pathways. Curr Opin Biotech 16, 174-179.
Oresic, M., Clish, C. B., Davidov, E. J., Verheij, E., Vogels, J. T. W. E., Havekes, L. M., Neumann, E., Adourian, A., Naylor, S., Greef, J. v. d. and Plasterer, T. (2004) Phenotype characterization using integrated gene transcript, protein and metabolite profiling. Appl. Bioinformatics 3, 205-217.
Ore\check{s}ic, M., Rischer, H. and Oksman-Caldentey, K. M. (2005) Metabolomics of plant secondary compounds: profiling of Catharanthus cell cultures. In Biotechnology in agriculture and forestry: plant metabolomics (Dixon, R., Willmitzer, L. and Saito, K.). Heidelberg: Springer,
Papon, N., Bremer, J., Vansiri, A., Andreu, F., Riedeau, M. and Creche, J. (2005) Cytokinin and ethylene control indole alkaloid production at the level of the MEP/terpenoid pathway in Catharanthus roseus suspension cells. Planta Med 71, 572-574.
Pasquali, G., Goddijn, O. J. M., Dewaal, A., Verpoorte, R., Schilperoort, R. A., Hoge, J. H. C. and Memelink, J. (1992) Coordinated regulation of two indole alkaloid biosynthetic genes from Catharanthus roseus by auxin and elicitors. Plant Mol Biol 18, 1121-1131.
Pauw, B., Hilliou, F. A. O., Martin, V. S., Chatel, G., de Wolf, C. J. F., Champion, A., Pre, M., van Duijn, B., Kijne, J. W., van der Fits, L. and Memelink, J. (2004) Zinc finger proteins act as transcriptional repressors of alkaloid biosynthesis genes in Catharanthus roseus. J Biol Chem 279, 52940-52948.
Rodriguez, S., Compagnon, V., Crouch, N. P., St-Pierre, B. and De Luca, V. (2003) Jasmonate-induced epoxidation of tabersonine by a cytochrome P-450 in hairy root cultures of Catharanthus roseus. Phytochemistry 64, 401-409.
Rozen, S, and Skaletsky, H. (2000) Primer3 on the WWW for general users and for biologist programmers. Methods Mol. Biol. 132, 365-386.
Schoendorf, A., Rithner, C. D., Williams, R. M. and Croteau, R. B. (2001) Molecular cloning of a cytochrome P450 taxane 10 beta-hydroxylase cDNA from Taxus and functional expression in yeast. P Natl Acad Sci USA 98, 1501-1506.
Schroder, G., Unterbusch, E., Kaltenbach, M., Schmidt, J., Strack, D., De Luca, V. and Schroder, J. (1999) Light-induced cytochrome P450-dependent enzyme in indole alkaloid biosynthesis: tabersonine 16-hydroxylase. Febs Lett 458, 97-102.
Schroder, G., Wehinger, E. and Schroder, J. (2002) Predicting the substrates of cloned plant O-methyltransferases. Phytochemistry 59, 1-8.
Siberil, Y., Benhamron, S., Memelink, J., Giglioli-Guivarc'h, N., Thiersault, M., Boisson, B., Doireau, P. and Gantet, P. (2001) Catharanthus roseus G-box binding factors 1 and 2 act as repressors of strictosidine synthase gene expression in cell cultures. Plant Mol Biol 45, 477-488.
St-Pierre, B. and Deluca, V. (1995) A cytochrome-P450 monooxygenase catalyzes the first step in the conversion of tabersonine to vindoline in Catharanthus roseus. Plant Physiol 109, 131-139.
St-Pierre, B., Vazquez-Flota, F. A. and De Luca, V. (1999) Multicellular compartmentation of Catharanthus roseus alkaloid biosynthesis predicts intercellular translocation of a pathway intermediate. Plant Cell 11, 887-900.
Swarup, R., Parry, G., Graham, N., Allen, T. and Bennett, M. (2002) Auxin cross-talk: integration of signalling pathways to control plant development. Plant Mol Biol 49, 411-426.
Tohge, T., Nishiyama, Y., Hirai, M. Y., Yano, M., Nakajima, J., Awazuhara, M., Inoue, E., Takahashi, H., Goodenowe, D. B., Kitayama, M., Noji, M., Yamazaki, M. and Saito, K. (2005) Functional genomics by integrated analysis of metabolome and transcriptome of Arabidopsis plants over-expressing an MYB transcription factor. Plant J 42, 218-235.
Toivonen, L., Ojala, M. and Kauppinen, V. (1991) Studies on the optimization of growth and indole alkaloid production by hairy root cultures of Catharanthus roseus. Biotechnol Bioeng 37, 673-680.
Urbanczyk-Wochniak, E., Luedemann, A., Kopka, J., Selbig, J., Roessner-Tunali, U., Willmitzer, L. and Fernie, A. R. (2003) Parallel analysis of transcript and metabolic profiles: a new approach in systems biology. Embo Rep 4, 989-993.
van der Fits, L. and Memelink, J. (2000) ORCA3, a jasmonate-responsive transcriptional regulator of plant primary and secondary metabolism. Science 289, 295-297.
van der Fits, L. and Memelink, J. (2001) The jasmonate-inducible AP2/ERF-domain transcription factor ORCA3 activates gene expression via interaction with a jasmonate-responsive promoter element. Plant J 25, 43-53.
van der Fits, L., Zhang, H., Menke, F. L. H., Deneka, M. and Memelink, J. (2000) A Catharanthus roseus BPF-1 homologue interacts with an elicitor-responsive region of the secondary metabolite biosynthetic gene Str and is induced by elicitor via a JA-independent signal transduction pathway. Plant Mol Biol 44, 675-685.
van der Heijden, R., Jacobs, D. I., Snoeijer, W., Hallard, D. and Verpoorte, R. (2004) The Catharanthus alkaloids: pharmacognosy and biotechnology. Curr Med Chem 11, 607-628.
Vandenabeele, S., van der Kelen, K., Boonefaes, T., Dat, J., Gadjev, I., Morsa, S., Rottiers, P., Zabeau, M., Inze, D. and van Breusegem, F. (2003) A comprehensive analysis of gene expression during H₂O₂ induced cell death in tobacco. Free Radical Res 37, 3-3.
Vazquez-Flota, F., Carrillo-Pech, M., Minero-Garcia, Y. and Miranda-Ham, M. D. (2004) Alkaloid metabolism in wounded Catharanthus roseus seedlings. Plant Physiol Bioch 42, 623-628.
Vazquez-Flota, F., De Luca, V., Carrillo-Pech, M., Canto-Flick, A. and Miranda-Ham, M. D. (2002) Vindoline biosynthesis is transcriptionally blocked in Catharanthus roseus cell suspension cultures. Mol Biotechnol 22, 1-8.
Vazquez-Flota, F. A. and De Luca, V. (1998) Jasmonate modulates development- and light-regulated alkaloid biosynthesis in Catharanthus roseus. Phytochemistry 49, 395-402.
Whitmer, S., van der Heijden, R. and Verpoorte, R. (2002) Effect of precursor feeding on alkaloid accumulation by a strictosidine synthase over-expressing transgenic cell line S1 of Catharanthus roseus. Plant Cell Tiss Org 69, 85-93.
Yahia, A., Kevers, C., Gaspar, T., Chenieux, J. C., Rideau, M. and Creche, J. (1998) Cytokinins and ethylene stimulate indole alkaloid accumulation in cell suspension cultures of Catharanthus roseus by two distinct mechanisms. Plant Sci 133, 9-15.
Zhao, J., Zhu, W. H. and Hu, Q. (2001) Enhanced catharanthine production in Catharanthus roseus cell cultures by combined elicitor treatment in shake flasks and bioreactors. Enzyme Microb Tech 28, 673-681.

1. An isolated polynucleotide that enhances the production of a compound selected from the group consisting of tabersonine, catharanthine, vinblastine, vincristine, and any combination thereof in Catharanthus roseus wherein said isolated polynucleotide is selected from the group consisting of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, any variant thereof, and any functional fragment thereof.

2. The isolated polynucleotide according to claim 1 wherein said polynucleotide shares at least 70% homology with a polynucleotide selected from the group consisting of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60; 61, 62, 63, 64, 65, and 66.

3. A recombinant DNA vector comprising at least one of the polynucleotide sequences according to claim 1.

4. A transgenic plant or a cell derived thereof that is transformed with a recombinant DNA vector according to claim 3.

5. A method of enhancing production in Catharanthus roseus of a compound selected from the group consisting of tabersonine, catharanthine, vinblastine, vincristine, and any combination thereof, the method comprising:
transforming the C. roseus with an expression vector comprising an expression cassette that comprises means for enhancing production, in Catharanthus roseus, of tabersonine, catharanthine, vinblastine, or vincristine so as to enhance the biosynthesis of tabersonine and/or catharanthine and/or vinblastine and/or vincristine in Catharanthus roseus.

6. The method according to claim 5, wherein the means for enhancing production, in C. roseus, of tabersonine, catharanthine, vinblastine, or vincristine is a polynucleotide selected from the group consisting of any one of SEQ ID NOs: 1 through 66, any variant of said selected polynucleotide SEQ ID NO, and any fragment thereof able to enhance production, in C. roseus, of tabersonine, catharanthine, vinblastine, or vincristine.

7. The method according to claim 6, wherein the polynucleotide has at least 70% sequence identity with a polynucleotide selected from the group consisting of any one of SEQ ID NOs: 1 through 66.

8. The recombinant DNA vector of claim 3, wherein the polynucleotide has at least 70% sequence identity with a polynucleotide selected from the group consisting of any one of SEQ ID NOs: 1 through 66.

9. A transgenic plant cell transformed with the recombinant DNA vector of claim 8.

10. A Catharanthus roseus plant cell transformed with a recombinant DNA vector, the recombinant DNA vector comprising an expression vector comprising an expression cassette, wherein the expression cassette is a DNA construct comprising:
a promoter region,
means for enhancing production, in C. roseus, of tabersonine, catharanthine, vinblastine, or vincristine, the means for enhancing production, in C. roseus, of tabersonine, catharanthine, vinblastine, or vincristine operatively associated with the promoter region, and
a termination sequence including a stop signal for RNA polymerase and a polyadenylation signal.

11. The transformed C. roseus plant cell of claim 10, wherein the means for enhancing production, in C. roseus, of tabersonine, catharanthine, vinblastine, or vincristine is a polynucleotide selected from the group consisting of any one of SEQ ID NOs: 1 through 66, any variant of said selected polynucleotide SEQ ID NO, and any fragment thereof able to enhance production, in C. roseus, of tabersonine, catharanthine, vinblastine, or vincristine.

12. The transformed C. roseus plant cell claim 11, wherein the polynucleotide has at least 70% sequence identity with a polynucleotide selected from the group consisting of any one of SEQ ID NOs: 1 through 66.


SEQUENCE LISTING (66 sequences)

SEQ ID NO: 1 - DNA, 215 bp, Catharanthus roseus
atgcgcaacaaatagggttggactactttcagatataactagggttcttacagaaaatgggttagcaattgtacgaacatatgtggcgacgcgcggagagaaggcggtgaatgctttctacgttagggatgtatcatgaaacaatgtggatatgcagtttgtgaagtcaatgaagagggaaatgggtgccattgatcaaatcgcagtaaagatta

SEQ ID NO: 2 - DNA, 197 bp, Catharanthus roseus
ttgctgtacctaaaaatggctgggctgctattagattcaaggcaaacaatcctggggtgtggttgctgcattgccacttggagagacacataagttggggaatggaaatggcattcatcattagaaatgggagacaccctgaccaaaaagttctgcctccacctccagacatgcctgcttgttactcaggactcatc

SEQ ID NO: 3 - DNA, 82 bp, Catharanthus roseus
tccaagttttctggaaagtgttataggtgataaaacaaaagctgtggctctcaaatggtgctaagaaggttactcaagactc

SEQ ID NO: 4 - DNA, 166 bp, Catharanthus roseus
gattcttgcatagtgaaattgacatgaaagggcggcattttgagctcattccatttggggcaggtaggagaatatgtccggggttacctctggcttatcggacgcttcatacaatgttggcaacttgtattcataactttgattggaagcttactcaggactcatc

SEQ ID NO: 5 - DNA, 107 bp, Catharanthus roseus
tgattccactctcttccatacttagatattccagttattgatatgagtcttttgacaacaaaggaagagctccaaaaactccactcagctttactcaggactcatca

SEQ ID NO: 6 - DNA, 112 bp, Catharanthus roseus
tatggtccgttggattgaagaagattcattggcatctaaagcacaaggaggaaccggagcggttcaattgagcaggtttctttttctaatggcgtttactcaggactcatca

SEQ ID NO: 7 - DNA, 66 bp, Catharanthus roseus
ccagattgcaaaggatatgggtgggaagaaaggttgggtggatcatttgtttactcaggactcatc

SEQ ID NO: 8 - DNA, 312 bp, Catharanthus roseus
aagtgtacggtgaggatgtagaggagttagatttgttggtgggtttgatggctgagaaaaagataaaaggcttcgctattagtgagactgcattcattattttcttgctcatggcaaccaggagattggaagcggatagattcttcacaagcaatttcaacgaggaaacatatacaaagaaaggtttagagtgggtgaacacaacggagagcttgaaagatgtgttggatcgtcactaccctgagatgacaaccaagtggatgaactccgacagtgctttttctgtgtgggatgcgcctcctcaatctgtta

SEQ ID NO: 9 - DNA, 151 bp, Catharanthus roseus
atttcaagcttcctttgccataagcaaccacaatgctgtcgagggttctcactccttcaatccaacccgggaatggttctttgtaagtgctagagacaattgttgggcgtatgataaccacttccaatttatccttactcaggactcatca

SEQ ID NO: 10 - DNA, 213 bp, Catharanthus roseus
ttttcaagtgagctattttgagtgtggaggagtggctattgctctatgcatatctcataagatcgctgatggctctacactatatacatttatgaataatttggctacttccacacgcctgtcatcacaggtgccaactccgttactgctttctgattccatttttccatgtggaaatgatatagttcctagaccattactcaggactcatca

SEQ ID NO: 11 - DNA, 117 bp, Catharanthus roseus
tgggaagctttgaacaagcaaaaaaagctgttgatgctggtgtagatgcgataattgtacaaggacgggaagcaggcgggcatgttattggacaggattcattactcaggactcatc

SEQ ID NO: 12 - DNA, 137 bp, Catharanthus roseus
atgatattcatcagcagttgtgaacttgtgcgggtccctaatgaagcttctgtcgataaatcttggattgggagaagaaattctacaaaatgcatttggaggggaggatatcggagcttgtttactcaggactcatc

SEQ ID NO: 13 - DNA, 100 bp, Catharanthus roseus
cacacattttctggactacttgggctaaccgtcctaccgaggcgcaatgttgcgaggtcaacggaaccatgtttcaaaatgttgttactcaggactcatc

SEQ ID NO: 14 - DNA, 99 bp, Catharanthus roseus
ggttccgttgactcgcacttgcgctcggtaggacggttagcccaagtagtccagaaaatgtgtgtgacgtcagtgagaggtgagatcactacgcagtca

SEQ ID NO: 15 - DNA, 113 bp, Catharanthus roseus
tttatttccaagagaatgtagggaagaatttgagatagatggaatgaccatacctaaaaaatcttgggtgattataaactactgggcaattggaagagatcactacgcagtca

SEQ ID NO: 16 - DNA, 212 bp, Catharanthus roseus
attgctgccatcaatagtatcatataaaatctgagctttcttcttattcttcttttcaacctcgaccaatccaccctgagccagcagatcatcaaaaactagcccacacatgtaaatcccaaaacaaggaggggtattgtacaaagaattgttttctgcgtgtatcttataatccaacataactggcgtaattggctgagcatttccaatta

SEQ ID NO: 17 - DNA, 296 bp, Catharanthus roseus
ggatacagtgcacagatttgctaaagaaattgtcaaacagaaaaaggaagaactcaaggtagaaatccaccatcgattccgtcgatctcttgtcacgattcctgaattccggccattcggacgaagatttcgtcaccgatattgtaatcagcttcatattagcaggtcgcgacactacatcctcggcattactcaggactcatcatcattactcaggactcatcatcattactcaggactcatcatcattactcaggactcatcatcattactcaggactcatcatcattactcag

SEQ ID NO: 18 - DNA, 215 bp, Catharanthus roseus
catattatccacccatccaatttcagtgcaatcttctttcgtcacacccaactcaggaaaacgttctttcatcaaggagagtaaggaatcaacatcaccttgaaaaaaacccatgaaggtggctattacagtcttgttacctgtttgatcattactcaggactcatcatcattactcaggactcatcatcattactcaggactcatcatcattac

SEQ ID NO: 19 - DNA, 164 bp, Catharanthus roseus
aacttgagcttctatgagtgttggaagggtgtgctcccatgaaacacgcattgatggcaattgtattatatgaccatcccaacaagcaatcattgaagacgattggggagattccatcccttgatgattttgtttacctcaatttgtagtgtattatttgttta

SEQ ID NO: 20 - DNA, 204 bp, Catharanthus roseus
cacacagtggcacatttgatgcataaattcaatattgagttgccaaatggagtcagtgctgaagattttgatcccacagatgccgttggactcgtatctcatgaccaaaaccctctctcgtttgtggcaactccggttaccattttttagtactttcactcggtgctaagtaaaaggttcaaatagtttactcaggactcatca

SEQ ID NO: 21 - DNA, 539 bp, Catharanthus roseus
tttccggagggtattgatatttactttgagaatgttggagggaagatgctggatgccgtccttctcaacatgagactccatggtcgtattgcagtatgtgggatgatctctcaatacaacctggatcagaacgaaggcgttcacaacctattctgcctaatcacgaagagaattcgaatggagggatttctggtgtttgattactatcatctgtacccgaaattcctggaggtgattctgccacaaataaaagaagggaaggtggtttacgtggaagacatagctgaaggacttgagaatgctccgagtgcactcataggcctattctcgggtcgtaatactgggaagcagctggtagttgttgcccctgaataaatgtacttactatcttcaatgctactattattgtatatttattgccctctgctctgagactcggagagactcttcttgctagtgtttctttctttatggaatgcaatgcggcaatgtgtatgaaattctatcaaggaaataatgttatgttgtgcttgcgcttt

SEQ ID NO: 22 - DNA, 213 bp, Catharanthus roseus
tttgtgacttgtgcaacatcactgtaaacaaaaatgcagtgttcggtgacagcagtgctttggcccctggaggcgttcgaattggtgctcccgccatgacatcgaggggtttggtcgagaaggacttctagcagattgccgagttcctacacgaggtctgttgaactttgcttgaagattcaaaaggagcatggaaaactcttgaaggacttt

SEQ ID NO: 23 - DNA, 119 bp, Catharanthus roseus
ctcttattgagattgggggcatatcgttcgcaagttgaaagaaagcaactagactaggaaataagaagagagagagaattctcatttatattattttgtccatttactcaggactcatc

SEQ ID NO: 24 - DNA, 306 bp, Catharanthus roseus
caagccattgatcccattatccaagaaagcatcaaggatactagttgcaggcacccatgctgataatattggcaatcagtgtggtggctggacaattgaatggcaaggtcttactggcaacatcacaaaaggtactacaatcctaactgccattagaaacactgtggataccaaaacagaagttgtatactcagaaaatccagatgctggatttgtgaaatcaaacaagtttgcctatgcaattgttgtggttggtgagccaccatattcagaaacctttggagattccttactcaggactcatca

SEQ ID NO: 25 - DNA, 176 bp, Catharanthus roseus
cacttttcttgaataaccagacattgagcttttatcatgttacccatcacagattcaagcagcaacagtaatggctattttggatgttttgtcaaatcattctccagatgaagaatatttgggaaaagaagcagaaggagcttgggctgaaaatcctattattactcaggactcat

SEQ ID NO: 26 - DNA, 283 bp, Catharanthus roseus
cagatggtctctttccggaacgacagctcttgtaacaggcgggactcgcgggatcggccgtgcaataatagaggagctagctgaattaggagcaaccatttacacttgttcaagaaatgaaacagagctcaatcaacttttgcaagattgtgcctcaaagggtttcaaagtttttgggtcaatctgtgatgtttcttccagagagcaaagaaaagggcttatggagaaagtttcctctgcatttgatggcaaactcaatatactaattactccaggactcatc

SEQ ID NO: 27 - DNA, 106 bp, Catharanthus roseus
gactcatcaagcccacagctaacgtcagccagcacctcaggatcactgtagtgagtaaccgcctgcacgattgccctagcccttctagctggatcactacacagtc

SEQ ID NO: 28 - DNA, 234 bp, Catharanthus roseus
ggaaaaaatgtgttgtgactgttggtgcttggatgaagaggctaattgagagagtaacaggggaaaaattgcctataaagccattggaaacaacagtacattattcggaaaatcaaaaaagggtatgaatcggagttcaagattgaaaatggtttcccaagttttgcaagttatggtgagccttacatctatggaaccccatctttggaattccctggtttactcaggactcat

SEQ ID NO: 29 - DNA, 139 bp, Catharanthus roseus
ttttcggaggatcataagcaactaatttggatgcaatttggacccaatttcatgtcaacaaggatgtttcaactctgctctccacaggattttgagctggcattgtctttggtgagaccacatcctggttctgttactc

SEQ ID NO: 30 - DNA, 203 bp, Catharanthus roseus
gagttgggcaccgcaattgaagatactgagtcatgaatcggtgggtggattcttgactcactcgggttggagttcggttgtggaggcaattcagtgtgaattagctctgattctgctgacattcttggctgaccaaggattgaatgcaagggttttggaggcaaagaagatggcttattccgtccccagagatgatcgagatg

SEQ ID NO: 31 - DNA, 169 bp, Catharanthus roseus
tgatgagtcctgaggtaacctgataaatcctcttggtgtttcttcagtgataagtccttctaaaggtccaatttcttgtccaactccgggtccccaaaacattaccacagagagtctttccttctctgagtttgtcacagctctgtgcaatgggatcactacgcagtca

SEQ ID NO: 32 - DNA, 253 bp, Catharanthus roseus
ccattagtacagccatacccaaaacaggcggcgtgccgacggtggtccaccttcacggtggaatccacccaccgcaaagcgacggaagcgctcttgcgtggttcacggcgaactacggcgaaaaaggcctgaaatggtctcaaatgacgtatatgtaccctaatgttcaacatgcaggaaatctttggtaccatgaccatgcccttggcttgactcgtgtgaaccttttggctggtttactcaggactcatca

SEQ ID NO: 33 - DNA, 54 bp, Catharanthus roseus
gcattagcttatgatagagccgcgtttcagatacgtggagctaaagctttactt

SEQ ID NO: 34 - DNA, 217 bp, Catharanthus roseus
gtataacgaatttcgctactagaagacaaagaggagtttgtattttgagtgggagcggcacggtaacaaatgttactctaaaacaaccagctgcaccgggtgcagtggtctcgttacatggaagattcgagattttatcactttccggttcgtttttgcctccaccggctccatcagccgcgtccggtttactcaggactcatcatttttttttttt

SEQ ID NO: 35 - DNA, 111 bp, Catharanthus roseus
ctcaatccattcttgcagtagtgatatcctctatagtttgatcaatttcagcttgtgtattcatcacaaatggaccatgctgcactactggttcattactcaggactcatc

SEQ ID NO: 36 - DNA, 332 bp, Catharanthus roseus
gcgtagtgatccaccaatgatgccctagctccttcctctgtcttatagacgaagaaggcaaaacctttagcctttccagtctgcttatcaaaccctaatggcccctcctcaatctccccatacattgaaaaatgagccaacagcctttctgaagatatctcgaatggaatattacctacgtaaatcttcctcaacgccacgtcagctgactgcgaattcccagaattgcctgctgctgcaagctgcgtaacggtaattcttccatcgattttcttattgggttctttgagtgccaaaatagcagcatcgatgtgtttactcaggactcatca

SEQ ID NO: 37 - DNA, 223 bp, Catharanthus roseus
tgaaagcaaaagtgcagtttgatttgatgcaattccagctaattgaacaattgttggactgtaatacattactgtatttattccaacaaattgcttgaaaaacttgaaggccgacgcctgccaccagccctcttcttactgattttgtttgtaatattttcacaatgtttatttttcctgaggatttgttttcttctatttcttgattactcaggactcatca

SEQ ID NO: 38 - DNA, 200 bp, Catharanthus roseus
ctgctcaaaaagcgcaagcttgaacttccgactattgaaaatgatgttgatgttgatgaagtcttttataacccaccgccaaaaggaagggttatcaagaaatatcatctaagtaatcaattcaggtgtctaaacaaagctgccctagtaactaaaggtgatttgaacccagatggatcaagagttgataaatggagatt

SEQ ID NO: 39 - DNA, 83 bp, Catharanthus roseus
agagcgtacagaaatcgagtgggcatctcggagtacagtatcagtcagtccactcttgaaaccatatttactcaggactcatc

SEQ ID NO: 40 - DNA, 377 bp, Catharanthus roseus
cacgatatgatagggcgatagaggtcacgtcgcttcagcatgaccttgaattgctccctggaggtgatcttactgaaattggtgaaagaggtgtaaacattagtggagggcaaaagcaaagagtgtccatggctagggctgtatattcaaattcagatgtttgcatattcgatgaccccctaagtgctcttgatgctcatgtggctcgtcaggttttcgaaaaatgcatcaagggagaactcagaggaaaaaccagagttcttgttaccaatcagctacactttctttcacaagtagataaaattatccttgtgcacgatggtatggtgaaagaggaaggaacctttgaagaactctcaaataatggtgtgctcttt

SEQ ID NO: 41 - DNA, 346 bp, Catharanthus roseus
tgacacagccatcaaaactggcatcgtcacgggcgtcatcgctcttgccgaaggaatagcagttggcagaagttttgcaatgttcaagaattacaatattgatggaaacaaagagatgattgcatttggaaccatgaacattcttggctctttcacctcttgttatctcaccactgggccattttcaagatcagcagtgaatttcaatgctggatgtaaaacagcagtatcaaacatagtaatggcattggcagtgatgataacattattgtttcttacaccacttttccattacacgcctctggtggtattatcatcaattataatagcagcaatgttgggatta

SEQ ID NO: 42 - DNA, 145 bp, Catharanthus roseus
tattactgaatcagaaatggcaatgcttattatgctgcttttcatactttgtgttctggtattggtattcaagctctcgttctccccgttgccttcaccttcctcggatggacatggggtatcatatgcttactcaggactcatc

SEQ ID NO: 43 - DNA, 155 bp, Catharanthus roseus
cttggggatggggtaatcccagcactcacattgatcatgggaggtaaccttttactcaggactcatacattagccaatcccggtaaagctgttgtagtgaggcataatccacaagtgtttcttgggaagaagagttgtttactcaggactcatca

SEQ ID NO: 44 - DNA, 162 bp, Catharanthus roseus
tgatgagtcctgaggtaagagctcccctgcttcttccttcaccttctcttctccttcgcctaccaccaagaaggttctccacagtgctggcctcaacttttcctccataaagttggcccaaaatcgcgcaatggctttctgataaggatcactacgcagtca

SEQ ID NO: 45 - DNA, 191 bp, Catharanthus roseus
gacttttctgagagtcatgggaattagcttgttttttagatgctttctcctttgccatttgctcgaacaagcttccatcataaacagctcgtatggtttctttgtgcagtagaccttgcttatccttgcagagcatatatagaatcttccattcagagtacgcagcaatccatcctttgtagtccttggga

SEQ ID NO: 46 - DNA, 481 bp, Catharanthus roseus
ccaaaacgaaacttatctgtcttggaaaattcacatgctcattctggtggtagatgcctttcagctgagtatacggatcgcgtcggtgagctagcaaagaagtatgggttgaagcttcacattgatggagctcgtatcttccatgcatcggttgctctgggagttcccgttcacaggcttgtgcaggctgctgattcagtctcggtatgtttatccaaaggccttggtgctccagtcggaagtgtgattgttggttcagaaagtttcattgccaaagcaaagattctcaggaagactttgggtggtggaatgaggcaggtcggtgtcctttgcgctgctgctcttgttgcatttcaagagaatgttggtaagctcgaagaagatcacaggaaggccaagactttagcagagggactgaacaaaatcaaaggactaaaagttgacattgattctgttgaaaccaacattgtatttttcgata

SEQ ID NO: 47 - DNA, 119 bp, Catharanthus roseus
gaatggaatgcaccatcacaatcatgtgctgcacgaacacaaccattctttggaacaaggtatccaacgggcacaccattatcatgggttataatgaacagagtattactcaggactca

SEQ ID NO: 48 - DNA, 237 bp, Catharanthus roseus
tgtatcaatatcttggtccgatcgcaaggaaggcatatgaagtggctatagaagatggcaagttcatttaccagcaaaccggccagcttcttgacacaagaggaggccctgaggataccaaatggatatttgtactcagtgtatcaatggtgttgtatgttgggcagaagaaaaaaggcacatttcaacattcaagcttcttggctggtggcgctactctgtctgctgggagattac

SEQ ID NO: 49 - DNA, 656 bp, Catharanthus roseus
cggtctcaaagatctaaatcttgttggaatcagtggaaaaacgggtgtttctgggaaatctaatgttacgatcccaacaacaatgaatttactcagcaatattggacatccgtgccctgttgattcccttcctcgattatccacggtggttcctgaattggatgcggcggcggcggcggaggagattaaaagatctcagatgactatattttacggaggtgaagttttggtgttccacgatttgccagccgacaaagctagagaagtaatgcaattggcaagcaacggaacctccagcatcggcagtaattcggcactcacaaaagccgagaaacaccattctcccgatgcctccctgccttccagttccaaggcaccgcccccaccaccggtttcttcaccagaaaggcctcctcaccctcaagtaccccaggctgttgaagctaacagttcagaattgccaattgcaagaagatcatcactgcataggtttcttgagaagagaaaagacagggctgttgctagaggaccatatcatcatcaaccagtacaaaaccaagcaccagcatcaacttcaacttcttccaagagtgatgaaccagaacaagaacaacttgaattgaaattataattttttgcctaattggttctgtttt

SEQ ID NO: 50 - DNA, 1223 bp, Catharanthus roseus
ctactttctctctctacacatatttcttcatacgcggaaatacatataaagtggtagcttcaaagatcagtattcttcaatcttcattcattcaggagttgtggttgtagctctgatttcagtgttcttgtggaaattcgtgatctgggtcggaaaaaaaatctggttttgttaattgtggaagaaaatcaatggcttcatcggagatgattatggattccggcagggcttctgctgttcagaagtctaacttttctcagacttgtagtcttttgagtcagtatttgaaggaaaacggtagttttggagatctcagccttggactcaaccgtaattttgaacccaatggggttcctgcaaaaactatgaatttgctatcaacgatggataaatctggccagaatttagaggctccggctgtgaaagagaacaaacctgggaatttgtttcctcagccggctggtttctacaccatccctgatataagtgttgcaaaatctgaaccagacacagcacaaatgaccatcttttatggtggacaagtcttggttttcaatgacttcccagctgaaaaggccagagaaatcatgcttttggcaagcaatggaagcccacttaatttcacccctaaaccagctgaatctgccaccggtttggtcactcctccaccccctgctgcatcaaatgttgtcccaagttttggaaatggtcttgtgcagcaggagaatgtcccaagccctctttatccacgcatcaatgatttaccactctcaagaaaagcatcacttaccaggttcttggagaagagaaaagataggattacagcaaaagcaccataccagatgatgaacaactattcttctaaggctgctgctgcttctgatcaaaaaggaacatggttgggttttggtcaacaatttcctgcaaatggaacgccaagtatagaaattggctttcctttttaaaacccctcaagaacttcttggggttggttgactctctctaccaagtttaagtttagatctaactatttatattcatcttttaatgtaattaaaattaaaaattcccaacgatctatctagttagtattatttctatattttcagatcttgaaaatgtatttttgaaggcatgtttccctacaatttcgtatgggagcataaattttattatatctgtttatttcctttcagatttcttgaaaaaaaaaaaaaaaaaaa

SEQ ID NO: 51 - DNA, 135 bp, Catharanthus roseus
tgaagagcccatcagccgcatttcttagattcttgttttcgttgtaaaaaacctcttggtgataacagagacatcttcatgtacaggtaattttgattgttccatttcggtttatttgttactcaggactcatca

SEQ ID NO: 52 - DNA, 95 bp, Catharanthus roseus
gatgatgctcttctatttcatgaagttgtaaggcaactgcaatgtgcttcaaaaactgaagttttggttcaggtaaaagttccttgatgcttcaa

SEQ ID NO: 53 - DNA, 1568 bp, Catharanthus roseus
tctatggcttaccatacgatgttccagattacgctagcttgggtggtcatatggccatggaggccccggggatccgaattcggcacgagaaagagagatagataagaaaggtgagataaatcatataaagagatcagtaaagtcggaaaaaatataataaattttcattgaaaaatagaaaaagacttgtatttctagtcggaagtagcaatatgatatcatagccaagttgtctgatattttggtatgcgcagatgtgctggactagttagtgtttgaataatgtttctcaaattttttagatttgctcaataccccatgaaaatctgggttataattaacagggttaaacaacatagtataagagatttttcttacatgttttgagaaatatggtacactgaatttcggaactttccatgttgggatctgagaaatattggatatggcagaggaatctcactcaggtaaggattcaaaaaccctcaaaatcttgagttacaacgtttgtttctgggatttggagttgcaagagagaatgaaagctataggccgcctcattcaattgcacaatccagatatcatttgctttcaggttccttatataaattacctctccttgtttagtacttaattatgaacttcttcttatatgtgattgctaatatataactgctgcttctgcttctgctgctgcgtgatcacattatacgaagtaacatcaagcatttacaacattttcaacaactccacttggtggaattcatataattgctcagtttcccttcagcagatcaagggacgttcatatttctgtatgcagctgagcatattgggagtaaaatcattcaacaccaaacccttcacaaattctagtatgggaagagaattatgcttagcagagatggaaatcagcagtggcagttctaatcctaaatcattaatcattgccacaagccatctggaaagtcctacccctgcacctccaacttttgatcaattaaacagcgaagaaagaatagaacaagcaaaagaatcaatcaaatttcttcaaaatgcccaaaacgtgattttctgcggtgatatgaattgggacgacgaatcagacggaaaatttccattacctaatgaatggtcagatgcttgggttgaattgaggccatggttgagaggttggacttatgacacaaaatctaatcaaatgttggttgcaaacaggccgttgcagaagagattggatcggtttttgaccaaattgaatgattttaaattggaaaagataagtatgattggtacagaggcgatccctggagtttcttacttgaaggagaaacgagtgaggaatagagtacagactttggagcttcctgttttgccaagtgatcattatgggttgatgttagaaattcacggtaagtaataaatgaaatgaaaatgtatggaaattattagggtttttatttcttcaagaaacactgcctctgttttgtacttagtatgtttgtggcttctggtatctgtcattttaatactaattaaatgataacttttatggaaaaaaaaaaaaa

SEQ ID NO: 54 - DNA, 479 bp, Catharanthus roseus
tttcgggatatgataacactcatgtatttgtttccatgatcagcatatggaacttcctaggtcgtattggcggtggtttcttttctgagaaaatcgtcagggactatgcttatcccagaccaatagctatcgccattgcccaagccataatggcaatcggacactttatcttcgctatgggttggcctggagagatgtacattggaactcttttggttggccttggctatggagctcactgggcaatcgtgccagctgctgcttcagaattgtttggcttgaaaaagtttggtgctctatataattttctcactctcgcaaaccctgccggctctttggtattctcaggtctcattgccagcagtatatatgacatagaagctgagaagcaagctcagcaacactatcctgcccaacaaatcacacattcagttttatcaagatttcttgctatggatgaaccattactcaggactcat

SEQ ID NO: 55 - DNA, 204 bp, Catharanthus roseus
ggaggatcacaacatctttagtttccatgggaggaacaacaggtgcagcttctggaggttttgctgcaactgcatttgtaaacatgtcccctggcatgattggaaatatgctatatggaagatcggctttctgattagcacatggggaatctgcagcagtaaagatcaaaaagttatttcctgatatttactcaggactcatca

SEQ ID NO: 56 - DNA, 74 bp, Catharanthus roseus
tgccttgtcccgcaggttccgatctctgccctgcttatagatagtccattttctcggaagatcactacgcagtc

SEQ ID NO: 57 - DNA, 398 bp, Catharanthus roseus
cggccagggtattaggatcaagaatattgaggcttggggtggggttatgggtcctgattacgactattttgagagaggaaacttggatattttcactggacgaggtccatgtttgaatgggccagtttgtgccatgaacttgacttccgatggtactggctcacatcccggctggtattgtaagtacgttgaggttacatttactggtgttcataagccctgtgctcagaaattcttctctgtggagcagtggttggcaaccgatgcgccgccgtatgagcttactgccaccaggaacaactgccgtaataagactgagatcggtcgctttgaatcttctccggtggtgtctgttgtgtaagaagctaaggtctatttatttcttggtctgattatta

SEQ ID NO: 58 - DNA, 118 bp, Catharanthus roseus
aactgtttttactgcgctgggctccaatgtatgcactgtaagctgtattggggcattgctagctgatggttctgtggttatttcagacgatttggctgtcattactcaggactcatca

SEQ ID NO: 59 - DNA, 252 bp, Catharanthus roseus
caattgctggaattgagaactagtgtgcttgatggaacagaatcatcgaaatgtttgcaaaagtcccagtctttccaaatccctcatgaattcctctgtccaataactctggagataatgacagaccctgttattgttgctactggacaggtaattctgaaattgaacgggttgacaaatataggtgtctcttgcagaaatattgtcaatcttgagaatgcagattatgaaatttctagctttttttgtctt

SEQ ID NO: 60 - DNA, 183 bp, Catharanthus roseus
tgatgagtcctgaggtaactagagatcaatatatgtcatttacatgtatgttgtataatgatgttcggggaaccaattacactatgataagggaaggaatgatctctacattgacattttgggactatgatttgaaactattgttggatagaaatgcatatttggtggatcactacgcagtca

SEQ ID NO: 61 - DNA, 69 bp, Catharanthus roseus
ctccaaccaaacttgcttgcatctgggctgatgaaggtgagatcagtatatgggatcactacgcagtca

SEQ ID NO: 62 - DNA, 788 bp, Catharanthus roseus
tctatggcttacccatacgatgttccagattacgctagcttgggtggtcatatggccatggaggccccggggatccgaattcggcacgagctcatttaccatggccgacgagaagcagcacctcttccaccaccaccaccaacaccacgaccaccatcacaaggaggaagtaaagcctgttgagggggttgcctactcagaaaccaagtatggctctgacggctcaggcggatatactgcagctactgaaactcttgttgtgtcagaagagccatctcatgattatgagaaagaaagaaagcaccacaagcatcttgagcagctcggagagcttggtgcccttgcttctggtgcctatgccctgcacgagaagcacgaggcaaagaaagatccagagcatgcccacaggcacaagatagaagaagagattgctgcagtagccggagttggtgcggcgggctttgcattccatgaacatcatgagaacaaagaatccaagaaggaagaagaaaaggctgaaggaaagcaccatcaccatctgttttaagctttcaaattgtgttttctttgatgatgatagaaacaatcatgtttcaataggataaataaacgtgttagcctgtctttctgtgtgattcgttcgggtttgactgttctccacattaatagcctttgcagttttaactttctaagtttttttttttaaataaaatgaagttgggctgtattttctatccaacaagaaaaaaaaaatttttagtttcattccttggatcaaatcgtttgag

SEQ ID NO: 63 - DNA, 991 bp, Catharanthus roseus
cacttttgtataacagaaaacgtcgtcgtaagaaagacttacattagaagaggatgtacgaaaacggtgattttgatgcagatttggctcttctcgattcaataaggcgacatttgctcggtgaatctctctctgaatctccgggaacattctccggtgaaagtcccaccggaatgtatggtggaagcggtaaggattcagaagaacaaatgcttatttatagtttccttaatgaagcaactactataaccgcagtgaaaatggagcctgaaaatactccaattgaatttcctcaatttttcaatttccaatcagtggcggcgccgccgccacaaaacgaaattaaaattaccagtccggaagtaaaagtagtagcggaaccaaagggaaaacattaccggggagtgagaaggcggccgtggggaaaattcgctgccgaaattagagatccagcgaagaacggagctcgtgtatggctaggaactttcgagacggcggaggatgctgccctagcttacgatcgagcagcttatcggatgagaggttcacgagcattgttgaattttcagttaaggataaattctggagaacctgaaccggttaggattacttcgaaaaggtctaattctaattcaaattcatcaacttcgtcgccgagtaattcgccttcgttgaagaggaagaaggctatcaaagtggcggagccgcctgtacttgttgtagagcaggatacggagcaccgtggctatggagaacagagattgtttagtgaatggtgactgcatttttcgattttttttttaatttattgggatttgtggaggattatacattatcaaatttcaaatttttttatttaatttcttgttcaaaaaaggaaagggaagaataatttcaaaatgtttgtatttgtgaaattaaattctctctctttcatgtaaacaaaaaaaaatttacaaaaatatctatcgattattttaaaaggattaag

SEQ ID NO: 64 - DNA, 618 bp, Catharanthus roseus
ttatccccaaataatgggtgctctttcgatcaattcccaaaattgcgctgtgaacaaatctcattaccatactcacaaattctttcttctttccaattacatcctattaggtgcagcttctagttgcattttcctaatcctttctctccgtttaatcccttccgtttccggtatcctcctaattctcctccacatcctcacgatcggcggtgctatttccggctgcagtgctgccgccggtggatctaacaaatggtacgctttacatatggtagcaactgtacttactgccatatttcaaggctctgtttcggtgcttatctttacaacaacgtctaatttcctcggcgcattgaaatcttatgttagagaagacgacggcgctttgattctgaaattggctggtggattgtcggttttgatcttctgtttggaatggattgtacttgttcttgcttttgtattgaggtattatggatttgttgaagggaatggaggaggtggggtttctgttagtaacagagttgttgaagatcaggggaaatgggcttctccatttcagccatttccatcccaagtttgatgatttttctcaagaaaagaagaaagccattgatt

SEQ ID NO: 65 - DNA, 1276 bp, Catharanthus roseus
cacctaaatagttagtaatatattacttctccaattctatacccacaaattttgagaaaaaattctacttcttaaagtatgaatatcagatcaaaaccacacttttgtgggtttttggtagaagaaacaaaagaagtttattagataggtcaatgtcttgcactgcaatggatttcttccctgcaaatttcatgttacaaacttctcatgaagatgaacatcctcatcatcaacatcctcccacttccctcagtactcccattctctctcaagattttcatggggttgcatcatttctagggaagagatctatgtcattttccggtgtggatatttgtgatcaagatggagtcggaggcggcggtggccacggaggagaggatgatttatcagatgatggatcacaattgggagagaaaaaaaggaggcttaatatggaacaagtgaagacacttgagaaaaactttgagttaggaaataagcttgaaccagaaaggaaaatgcagttggctagggctcttggtttgcagcctagacaaattgctatttggtttcaaaacagaagggcaagatggaaaactaaacaattggaaaaagattatgatattttaaaaagacaatttgaagctattaaagctgaaaatgatgcacttcaagcccaaaaccaaaaacttcatgctgagataatggcactaaaaaatagggaaccgactgaatcgatcaacctgaacaaagaaacagaaggatcttgcagtaacagaagtgaaaacagttctgataatatcaaattggacatttcaagaacaacaccggctatagacagtcacccacaaaccagcagacctttttttccatcttctcttataaggccaaacacggcacaacaacttttccaaactacttcatcttcatcaagaccaccacctgctgatcatcatcacttaatccaatgccagaataataataataataatccaacggctactgttaaagaagagagcagtttgtgcaatatgttttgtaccattgatgatcaaaccggattttggccttggttggagcagcaccatttcaattgatgatgaattccgggatcggatcgggtcgggttttaaattttaatttactcttttttttttttatttggaaaattagaaggaccaaagggccctttgattgaccaaaaaaaatatttaccaggaaattaattactggcgggtatttttttttgggatttaattttaaaaaattttttgtggtaaaaagaaaaaaaaaa

SEQ ID NO: 66 - DNA, 97 bp, Catharanthus roseus
tgactgcgtagtgatccaagtggtcataactttctctgctaccgcttttgcaactattgggatgatcataaacaacgattttactcaggactcatca